SEQUENCE LISTING (2 sequences)

SEQ ID NO: 1 - DNA, 19 bp, synthetic construct
  source 1..19: mol_type = other DNA
gtgccagcmgccgcggtaa

SEQ ID NO: 2 - DNA, 20 bp, synthetic construct
  source 1..20: mol_type = other DNA
ggactachvgggtwtctaat

(19) United States
(12) Patent Application Publication
(10) Pub. No.: US 2026/0085364 A1
(43) Pub. Date: Mar. 26, 2026

(54) USE OF BIOMARKERS IN TREATMENT WITH BIFIDOBACTERIA

(71) Applicant: International N&H Denmark ApS, Kongens Lyngby (DK)

(72) Inventors: Niels Christensen, Aarthus C (DK); Henrik Max Jensen, Brabrand (DK); Henna-Maria Kailanto, Kirkkonummi (FI); Christian Clement Yde, Aarhus (DK)

(21) Appl. No.: 19/112,320

(22) PCT Filed: Sep. 21, 2023
(86) PCT No.: PCT/EP2023/076064

§ 371 Date: Mar. 17, 2025

(30) Foreign Application Priority Data
Sep. 22, 2022 (EP) 22197060.1

(51) Int. Cl. C12Q 1/689; G01N 33/53; G01N 33/92

(52) U.S. Cl. CPC C12Q 1/689; G01N 33/5308; G01N 33/92; C12Q 2600/106; G01N 2570/00

(57) ABSTRACT

This invention relates to a method of identifying a subject having an increased probability of having a beneficial clinical response to administration of a bacterial strain of the genus Bifidobacterium or a mixture of two or more strains thereof to said subject, by measuring the level of some specific biomarkers in said subject.

### FIELD OF THE INVENTION

This invention relates to a method of identifying a subject having an increased probability of having a beneficial clinical response to administration of a bacterial strain of the genus Bifidobacterium or a mixture of two or more strains thereof to said subject, by measuring the level of some specific biomarkers in said subject.

### BACKGROUND OF THE INVENTION

Regulation of energy balance is critical for the survival of an organism. When nutrients are freely available, they are stored to account for low energy intake during times of scarcity. In a normal state the brain, together with energy storage tissues, regulates energy balance by reducing energy intake when energy stores are congested. If the energy storage machinery is disturbed, the brain is no longer able to maintain energy balance. This could lead to inability to maintain adequate energy intake, often manifested as wilting in elderly populations, or to excess storage of energy in adipose tissue and even obesity.

Body mass index (BMI) is a simple index of weight-for-height that is commonly used to classify body weight status in adults. It is defined as a person's weight in kilograms divided by the square of his height in meters (kg/m2).

The World Health Organisation (WHO) defines a BMI below 18.5 as underweight; a BMI between 18.5 and 24.99 as normal weight; a BMI greater than or equal to 25 as overweight; a BMI greater than or equal to 30 as obesity.

Overweight and obesity are defined as abnormal or excessive fat accumulation. Underweight is defined as a body weight that is too low to maintain normal bodily functions. Both underweight and overweight may lead to disturbances in metabolic functions, such as hormonal signalling.

Once considered a high-income country problem, overweight and obesity are now on the rise in low- and middle-income countries, particularly in urban settings. In developing countries with emerging economies (classified by the World Bank as lower- and middle-income countries) the rate of increase of childhood overweight and obesity has been more than 30% higher than that of developed countries.

Overweight and obesity are linked to more deaths worldwide than underweight. Most of the world's population live in countries where overweight and obesity kill more people than underweight (this includes all high-income and most middle-income countries). The fundamental cause of obesity and overweight is an energy imbalance between calories consumed and calories expended.

The most common consequences of overweight and obesity are diseases such as: cardiovascular diseases (mainly heart disease and stroke), [which were the leading cause of death in 2012]; diabetes; musculoskeletal disorders (especially osteoarthritis—a highly disabling degenerative disease of the joints); some cancers (endometrial, breast, and colon).

The risk for these diseases increases with an increase in BMI.

Childhood obesity is associated with a higher chance of obesity, premature death and disability in adulthood. But in addition to increased future risks, obese children experience breathing difficulties, increased risk of fractures, hypertension, early markers of cardiovascular disease, insulin resistance and psychological effects.

Overweight and obesity, as well as their related diseases, are largely preventable, and the food industry can play a significant role in the fight against obesity.

It is well known that dysfunctional energy regulation may lead to a variety of metabolic disorders, including obesity. The connections between gut microbiota, energy homeostasis, and the pathogenesis of metabolic disorders are now well-established (Amabebe et al. 2020).

The publication “Neuronal regulation of Energy Homeostasis: Beyond the Hypothalamus and feeding”, by Waterson and Horvath 2015, stresses the importance of the brain in maintaining energy homeostasis and its relationship with obesity and other metabolic diseases.

Probiotics are live microorganisms that confer a health benefit on the host when administered in adequate amounts, whereas prebiotics are substrates that are selectively utilised by host microorganisms conferring a health benefit. Preliminary evidence shows that oral administration of certain probiotics in clinical intervention studies significantly impacts body composition or weight management (Stenman et al., 2016), which suggests a link between the gut microbiota and body fat regulation in humans. It has been shown that the probiotic strain Bifidobacterium animalis ssp. lactis 420 (B420) reduces the accumulation of body fat in humans (Stenman et al., 2016).

Given the potential of some probiotics and prebiotics to improve weight management within the wider population, it is important to be able to better predict which subjects among the wider population will respond better to the administration of such probiotics and prebiotics.

The present invention seeks to provide a solution to the problems of the prior art.

### SUMMARY OF THE INVENTION

In one aspect, the invention concerns a method of identifying a subject having an increased probability of having a beneficial clinical response to administration of a bacterial strain of the genus Bifidobacterium or a mixture of two or more strains thereof, the method comprising the steps of,
i. measuring the level of at least one of the following biomarkers: hPAI1 Total, the bile acid GLCA, the bile acid LCA, the bacteria Coprococcus, the bacteria Ruminococcus and/or the bacteria Akkermansia, in a biological sample obtained from said subject, and
ii. comparing said level to a threshold value,
   wherein when the measured level of at least one of the biomarkers is higher than said threshold value, the subject is identified as having said increased probability.

In another aspect, the present invention relates to a method of identifying a subject having an increased probability of having a beneficial clinical response to administration of a bacterial strain of the genus Bifidobacterium or a mixture of two or more strains thereof, the method comprising the steps of,
i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps or corresponding physical activity; and
ii. comparing said level to a threshold value,
   wherein when the measured level of at least one of the biomarkers is higher than said threshold value, the subject is identified as having said increased probability.

In another aspect, the present invention relates to a use of a bacterial strain of the genus Bifidobacterium or a mixture of two or more strains thereof for at least one of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject, wherein said subject has been identified as having an increased probability of having a beneficial clinical response to administration of said bacterial strain of the genus Bifidobacterium or a mixture of two or more strains thereof, by,
i. measuring the level of at least one of the following biomarkers: hPAI1 Total, the bile acid GLCA, the bile acid LCA, the bacteria Coprococcus, the bacteria Ruminococcus and/or the bacteria Akkermansia, in a biological sample obtained from said subject, and
ii. comparing said level to a threshold value, wherein when the measured level of at least one of the biomarkers is higher than said threshold value, the subject is identified as having said increased probability.

In another aspect, the present invention relates to a use of a bacterial strain of the genus Bifidobacterium or a mixture of two or more strains thereof for at least one of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject, wherein said subject has been identified as having an increased probability of having a beneficial clinical response to administration of said bacterial strain of the genus Bifidobacterium or a mixture of two or more strains thereof, by,
i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps or corresponding physical activity; and
ii. comparing said level to a threshold value,
   wherein when the measured level of at least one of the biomarkers is higher than said threshold value, the subject is identified as having said increased probability.

In a further aspect, the present invention relate to bacterial strain of the genus Bifidobacterium or a mixture of two or more strains thereof for use in managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject, wherein said subject has been identified as having an increased probability of having a beneficial clinical response to administration of said bacterial strain of the genus Bifidobacterium or a mixture of two or more strains thereof, by,
i. measuring the level of at least one of the following biomarkers: hPAI1 Total, the bile acid GLCA, the bile acid LCA, the bacteria Coprococcus, the bacteria Ruminococcus and/or the bacteria Akkermansia, in a biological sample obtained from said subject, and
ii. comparing said level to a threshold value,
   wherein when the measured level of at least one of the biomarkers is higher than said threshold value, the subject is identified as having said increased probability.

In a further aspect, the present invention relate to bacterial strain of the genus Bifidobacterium or a mixture of two or more strains thereof for use in managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject, wherein said subject has been identified as having an increased probability of having a beneficial clinical response to administration of said bacterial strain of the genus Bifidobacterium or a mixture of two or more strains thereof, by,
i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps or corresponding physical activity; and
ii. comparing said level to a threshold value,
   wherein when the measured level of at least one of the biomarkers is higher than said threshold value, the subject is identified as having said increased probability.

In another aspect, the present invention relates to a method of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject in need thereof, said method comprising administering a bacterial strain of the genus Bifidobacterium or a mixture of two or more strains thereof to said subject, wherein said subject has been identified as having an increased probability of having a beneficial clinical response to the administration of said bacterial strain of the genus Bifidobacterium or a mixture of two or more strains thereof, by,
i. measuring the level of at least one of the following biomarkers: hPAI1 Total, the bile acid GLCA, the bile acid LCA, the bacteria Coprococcus, the bacteria Ruminococcus and/or the bacteria Akkermansia, in a biological sample obtained from said subject, and
ii. comparing said level to a threshold value,
   wherein when the measured level of at least one of the biomarkers is higher than said threshold value, the subject is identified as having said increased probability. In another aspect, the present invention relates to a method of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject in need thereof, said method comprising administering a bacterial strain of the genus Bifidobacterium or a mixture of two or more strains thereof to said subject, wherein said subject has been identified as having an increased probability of having a beneficial clinical response to the administration of said bacterial strain of the genus Bifidobacterium or a mixture of two or more strains thereof, by,
i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps or corresponding physical activity; and
ii. comparing said level to a threshold value,
   wherein when the measured level of at least one of the biomarkers is higher than said threshold value, the subject is identified as having said increased probability.

In another aspect, the present invention relates to a method of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject in need thereof, said method comprising
i. Prescribing to the subject a daily personal activity of more than 7000 steps, or corresponding physical activity; and
ii. administering a bacterial strain of the genus Bifidobacterium or a mixture of two or more strains thereof to the subject having obtained the activity under i), which subject has an increased probability of having a beneficial clinical response to the administration of said bacterial strain of the genus Bifidobacterium or a mixture of two or more strains thereof.

In some specific embodiments this bacterial strain of the genus Bifidobacterium or a mixture of two or more strains thereof is as described herein.

### DESCRIPTION OF FIGURES

FIG. 1. Human subject BMI (kg/m2) Visit 8 minus Visit 2 plotted against amount of hPAI1 Total levels (pg/mL) in blood. Grey markers and linear trendline is data from B420 and black are data from the Placebo group. Dotted line is the threshold.

FIG. 2. Human subject BMI (kg/m2) Visit 8 minus Visit 2 plotted against amount of Glucolithocholic acid levels (GLCA, μmol/L) in blood. Grey markers and linear trendline is data from B420 and black are data from the Placebo group. Dotted line is the threshold.

FIG. 3. Human subject BMI (kg/m2) Visit 8 minus Visit 2 plotted against amount of Lithocholic acid levels (LCA, μmol/L) in blood. Grey markers and linear trendline is data from B420 and black are data from the Placebo group. Dotted line is the threshold.

FIG. 4. Human subject BMI (kg/m2) Visit 8 minus Visit 2 plotted against relative abundance of Coprococcus. Grey markers and linear trendline is data from B420 and black are data from the Placebo group. Dotted line is the threshold.

FIG. 5. Human subject BMI (kg/m2) Visit 8 minus Visit 2 plotted against relative abundance of Ruminococcus. Grey markers and linear trendline is data from B420 and black is data from the Placebo group. Dotted line is the threshold.

FIG. 6. Human subject BMI (kg/m2) Visit 8 minus Visit 2 plotted against relative abundance of Akkermansia. Grey markers and linear trendline is data from B420 and black are data from the Placebo group. Dotted line is the threshold.

FIG. 7. Human subject Trunk Fat(g) by DXA-scan Visit 8 minus Visit 2 plotted against hPAI1 Total. Grey markers and linear trendline is data from B420 and black are data from the Placebo group. Dotted line is the threshold.

FIG. 8. Human subject Trunk Fat (g) by DXA-scan Visit 8 minus Visit 2 plotted against amount of Glucolithocholic acid. Grey markers and linear trendline is data from B420 and black are data from the Placebo group. Dotted line is the threshold.

FIG. 9. Human subject Trunk Fat(g) by DXA-scan Visit 8 minus Visit 2 plotted against amount of Lithocholic acid. Grey markers and linear trendline is data from B420 and black are data from the Placebo group. Dotted line is the threshold.

FIG. 10. Human subject Trunk Fat(g) by DXA-scan Visit 8 minus Visit 2 plotted against relative abundance of Coprococcus. Grey markers and linear trendline is data from B420 and black are data from the Placebo group. Dotted line is the threshold.

FIG. 11. Human subject Trunk Fat(g) by DXA-scan Visit 8 minus Visit 2 plotted against relative abundance of Ruminococcus. Grey markers and linear trendline is data from B420 and black are data from the Placebo group. Dotted line is the threshold.

FIG. 12. Human subject Trunk Fat (g) by DXA-scan Visit 8 minus Visit 2 plotted against relative abundance of Akkermansia. Grey markers and linear trendline is data from B420 and black are data from the Placebo group. Dotted line is the threshold.

FIG. 13. Human subject Android fat (g) by DXA scan Visit 8 minus Visit 2 plotted against amount of hPAI1 Total. Grey markers and linear trendline is data from B420 and black are data from the Placebo group. Dotted line is the threshold.

FIG. 14. Human subject Android fat (g) by DXA scan Visit 8 minus Visit 2 plotted against amount of Glucolithocholic acid. Grey markers and linear trendline is data from B420 and black are data from the Placebo group. Dotted line is the threshold.

FIG. 15. Human subject Android fat(g) by DXA scan Visit 8 minus Visit 2 plotted against amount of Lithocholic acid. Grey markers and linear trendline is data from B420 and black are data from the Placebo group. Dotted line is the threshold.

FIG. 16. Human subject Android fat(g) by DXA scan Visit 8 minus Visit 2 plotted against relative abundance of Coprococcus. Grey markers and linear trendline is data from B420 and black are data from the Placebo group. Dotted line is the threshold.

FIG. 17. Human subject Android fat (g) by DXA scan Visit 8 minus Visit 2 plotted against relative abundance of Ruminococcus. Grey markers and linear trendline is data from B420 and black are data from the Placebo group. Dotted line is the threshold.

FIG. 18. Human subject Android fat (g) by DXA scan Visit 8 minus Visit 2 plotted against relative abundance of Akkermansia. Grey markers and linear trendline is data from B420 and black are data from the Placebo group. Dotted line is the threshold.

FIG. 19. Human subject Total fat(g) by DXA scan Visit 8 minus Visit 2 plotted against amount of hPAI1 Total. Grey markers and linear trendline is data from B420 and black are data from the Placebo group. Dotted line is the threshold FIG. 20. Human subject Total fat(g) by DXA scan Visit 8 minus Visit 2 plotted against amount of Glucolithocholic acid. Grey markers and linear trendline is data from B420 and black are data from the Placebo group. Dotted line is the threshold.

FIG. 21. Human subject Total fat (g) by DXA scan Visit 8 minus Visit 2 plotted against amount of Lithocholic acid. Grey markers and linear trendline is data from B420 and black are data from the Placebo group. Dotted line is the threshold.

FIG. 22. Human subject Total fat (g) by DXA scan Visit 8 minus Visit 2 plotted against relative abundance of Coprococcus. Grey markers and linear trendline is data from B420 and black are data from the Placebo group. Dotted line is the threshold.

FIG. 23. Human subject Total fat (g) by DXA scan Visit 8 minus Visit 2 plotted against relative abundance of Ruminococcus. Grey markers and linear trendline is data from B420 and black are data from the Placebo group. Dotted line is the threshold.

FIG. 24. Human subject Total fat (g) by DXA scan Visit 8 minus Visit 2 plotted against relative abundance of Akkermansia. Grey markers and linear trendline is data from B420 and black is data from the Placebo group. Dotted line is the threshold.

FIG. 25. Plot of DXAFMASS (v5-v2) vs relative amount Lipid_ESP PC(36:4) in serum at v2 for both B420 and Placebo treatments. Prospectively, B420 has a lower slope compared to Placebo, meaning that Lipid_ESP PC(36:4) in high values increases the probability of total fat reduction for B420 treatment.

Retrospectively, a high value of Lipid_ESP PC(36:4) for B420 treatment is associated to total fat loss for responders but not for non-responders. Dotted line is the threshold.

FIG. 26. Plot of DXAFMASS (v5-v2) vs relative amount Lipid_ESP PC(32:1) in serum at v2 for both B420 and Placebo treatments. Prospectively, B420 has a lower slope compared to Placebo, meaning that Lipid_ESP PC(32:1) in high values increases the probability of total fat reduction for B420 treatment.

Retrospectively, a high value of Lipid_ESP PC(32:1) for B420 treatment is associated to total fat loss for responders but not for non-responders. Dotted line is the threshold.

FIG. 27. Plot of DXAFMASS (v5-v2) vs relative amount Lipid_ESP PE(38:4) in serum at v2 for both B420 and Placebo treatments. Prospectively, B420 has a lower slope compared to Placebo, meaning that Lipid_ESP PE(38:4) in high values increases the probability of total fat reduction for B420 treatment. Retrospectively, a high value of Lipid_ESP PE(38:4) for B420 treatment is associated to total fat loss for responders but not for non-responders. Dotted line is the threshold.

FIG. 28. Plot of DXAFMASS (v5-v2) vs relative amount Lipid_ESP SM(d40:1) in serum at v2 for both B420 and Placebo treatments. Prospectively, B420 has a lower slope compared to Placebo, meaning that Lipid_ESP SM(d40:1) in high values increases the probability of total fat reduction for B420 treatment.

Retrospectively, a high value of Lipid_ESP SM(d40:1) for B420 treatment is associated to total fat loss for responders but not for non-responders. Dotted line is the threshold.

FIG. 29. Plot of DXAFMASS (v5-v2) vs relative amount Lipid_ESN PE(36:4) in serum at v2 for both B420 and Placebo treatments. Prospectively, B420 has a lower slope compared to Placebo, meaning that Lipid_ESN PE(36:4) in high values increases the probability of total fat reduction for B420 treatment.

Retrospectively, a high value of Lipid_ESN PE(36:4) for B420 treatment is associated to total fat loss for responders but not for non-responders. Dotted line is the threshold.

FIG. 30. Plot of DXAFMASS (v5-v2) vs relative amount Lipid_ESN PI(40:4) in serum at v2 for both B420 and Placebo treatments. Prospectively, B420 has a lower slope compared to Placebo, meaning that Lipid_ESN PI(40:4) in high values increases the probability of total fat reduction for B420 treatment.

Retrospectively, a high value of Lipid_ESN PI(40:4) for B420 treatment is associated to total fat loss for responders but not for non-responders. Dotted line is the threshold.

FIG. 31. Plot of DXAFMASS (v5-v2) vs relative amount Lipid_ESN PE(40:5) in serum at v2 for both B420 and Placebo treatments. Prospectively, B420 has a lower slope compared to Placebo, meaning that Lipid_ESN PE(40:5) in high values increases the probability of total fat reduction for B420 treatment.

Retrospectively, a high value of Lipid_ESN PE(40:5) for B420 treatment is associated to total fat loss for responders but not for non-responders. Dotted line is the threshold.

FIG. 32. Plot of DXAFMASS (v5-v2) vs BA(ISO-LCA) in feces at v2 for both B420 and Placebo treatments. Prospectively, B420 has a lower slope compared to Placebo, meaning that vs BA(ISO-LCA) in high values increases the probability of total fat reduction for B420 treatment.

Retrospectively, a high value of vs BA(ISO-LCA) for B420 treatment is associated to total fat loss for responders but not for non-responders. Dotted line is the threshold.

FIG. 33. Plot of DXAFMASS (v5-v2) vs BA(LCA) in feces at v2 for both B420 and Placebo treatments. Prospectively, B420 has a lower slope compared to Placebo, meaning that BA(LCA) in high values increases the probability of total fat reduction for B420 treatment. Retrospectively, a high value of BA(LCA) for B420 treatment is associated to total fat loss for responders but not for non-responders. Dotted line is the threshold.

FIG. 34. Plot of DXAFMASS (v5-v2) vs relative abundance Taxa (Bacteria_Firmicutes_Clostridia_Clostridiales_Clostridiales_Incertae Sedis XIII_Aminipila_Aminipila butyrica) in feces at v2 for both B420 and Placebo treatments.

Prospectively, B420 has a lower slope compared to Placebo, meaning that (Bacteria_Firmicutes_Clostridia_Clostridiales_Clostridiales_Incertae Sedis XIII_Aminipila_Aminipila butyrica) in high values increases the probability of total fat reduction for B420 treatment.

Retrospectively, a high value of (Bacteria_Firmicutes_Clostridia_Clostridiales_Clostridiales_Incertae Sedis XIII_Aminipila_Aminipila butyrica) for B420 treatment is associated to total fat loss for responders but not for non-responders. Dotted line is the threshold.

FIG. 35. Plot of GLUC (v5-v2) vs relative abundance Taxa (Bacteria_Firmicutes_Clostridia_Clostridiales_Clostridiales_Incertae Sedis XIII_Unclassified Clostridiales_Incertae Sedis XIII_Unclassified Clostridiales_Incertae Sedis XIII) in feces at v2 for both B420 and Placebo treatments. Prospectively, B420 has a lower slope compared to Placebo, meaning that (Bacteria_Firmicutes_Clostridia_Clostridiales_Clostridiales_Incertae Sedis XIII_Unclassified Clostridiales_Incertae Sedis XIII_Unclassified Clostridiales_Incertae Sedis XIII) in high values increases the probability of glucose reduction for B420 treatment.

Retrospectively, a high value of (Bacteria_Firmicutes_Clostridia_Clostridiales_Clostridiales_Incertae Sedis XIII_Unclassified Clostridiales_Incertae Sedis XIII_Unclassified Clostridiales_Incertae Sedis XIII) for B420 treatment is associated to glucose lowering for responders but not for non-responders. Dotted line is the threshold.

FIG. 36. Plot of GLUC (v5-v2) vs relative abundance Taxa (Bacteria_Firmicutes_Clostridia_Clostridiales_Eubacteriaceae_Eubacterium_Eubacterium coprostanoligenes) in feces at v2 for both B420 and Placebo treatments. Prospectively, B420 has a lower slope compared to Placebo, meaning that (Bacteria_Firmicutes_Clostridia_Clostridiales_Eubacteriaceae_Eubacterium_Eubacterium coprostanoligenes) in high values increases the probability of glucose reduction for B420 treatment.

Retrospectively, a high value of (Bacteria_Firmicutes_Clostridia_Clostridiales_Eubacteriaceae_Eubacterium_Eubacterium coprostanoligenes) for B420 treatment is associated to glucose lowering for responders but not for non-responders. Dotted line is the threshold.

FIG. 37. Plot of GLUC (v5-v2) vs relative response (rel. to total peak area) of metabolite Pimelic acid in feces at v2 for both B420 and Placebo treatments. Prospectively, B420 has a lower slope compared to Placebo, meaning that (Pimelic acid) in high values increases the probability of glucose reduction for B420 treatment.

Retrospectively, a high value of (Pimelic acid) for B420 treatment is associated to glucose lowering for responders but not for non-responders. Dotted line is the threshold.

FIG. 38. Plot of GLUC (v5-v2) vs relative response (rel to total peak area) of metabolite Azelaic acid in feces at v2 for both B420 and Placebo treatments. Prospectively, B420 has a lower slope compared to Placebo, meaning that (Azelaic acid) in high values increases the probability of glucose reduction for B420 treatment.

Retrospectively, a high value of (Azelaic acid) for B420 treatment is associated to glucose lowering for responders but not for non-responders. Dotted line is the threshold.

FIG. 39. Plot of GLUC (v5-v2) vs STP (AVGSTEPS) in clinical study at v2 for both B420 and Placebo treatments. Prospectively, B420 has a lower slope compared to Placebo, meaning that (AVGSTEPS) in high values increases the probability of glucose reduction for B420 treatment.

Retrospectively, a high value of (AVGSTEPS) for B420 treatment is associated to glucose lowering for responders but not for non-responders. Dotted line is the threshold.

FIG. 40. Plot of GLUC (v5-v2) vs BA (DCA) in serum at v2 for both B420 and Placebo treatments. Prospectively, B420 has a lower slope compared to Placebo, meaning that BA (DCA) in high values increases the probability of glucose reduction for B420 treatment.

Retrospectively, a high value of BA (DCA) for B420 treatment is associated to glucose lowering for responders but not for non-responders. Dotted line is the threshold.

### DETAILED DISCLOSURE OF THE INVENTION

### Advantages

It has surprisingly been found by the present inventors that measuring the level of one or more of the biomarkers hPAI1 Total, GLCA, LCA, Coprococcus, Ruminococcus and Akkermansia at baseline in a subject may predict if said subject will have a beneficial clinical response to administration of a bacterial strain of the genus Bifidobacterium or a mixture of two or more said strains thereof, in particular strain Bifidobacterium animalis subsp. lactis 420 (strain B420), compared to a placebo group.

The detailed aspects of this invention are set out below. In part some of the detailed aspects are discussed in separate sections. This is for ease of reference and is in no way limiting. All of the embodiments described below are equally applicable to all aspects of the present invention unless the context specifically dictates otherwise.

### Bacteria

The bacterium used in the present invention is selected from a bacterium of the genus Bifidobacterium or a mixture thereof. Preferably the Bifidobacterium to be used in the present invention is a Bifidobacterium which is generally recognised as safe and, which is preferably GRAS approved. Generally recognized as safe (GRAS) is an American Food and Drug Administration (FDA) designation that a chemical or substance added to food is considered safe by experts, and so is exempted from the usual Federal Food, Drug, and Cosmetic Act (FFDCA) food additive tolerance requirements.

In particular, the bacterial strain is Bifidobacterium animalis ssp. lactis 420 (B420).

The bacterial strains are commercially available from DuPont Nutrition Biosciences Aps of Denmark.

The bacterial strain B420 was also deposited by DuPont Nutrition Biosciences Aps of Denmark, in accordance with the Budapest Treaty on the international recognition of the deposit of microorganisms for the purposes of patent procedure at the Leibniz-Institut Deutsche Sammlung von Mikroorganismen und Zellkulturen GmbH (DSMZ), Inhoffenstrasse 7B, 38124 Braunschweig, Germany, where it is recorded under the following registration number:

Strain B420 (DGCC420); deposited on 30 Jun. 2015 under registration number DSM32073.

In a first aspect, the invention relates to a method of identifying a subject having an increased probability of having a beneficial clinical response to administration of a bacterial strain of the genus Bifidobacterium or a mixture of two or more strains thereof, the method comprising the steps of,
i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps or corresponding physical activity; and
ii. comparing said level to a threshold value,
   wherein when the measured level of at least one of the biomarkers is higher than said threshold value, the subject is identified as having said increased probability.

In the context of the present invention, a biomarker, or biological marker, is a measurable indicator of some biological state or condition of a subject. Biomarkers are measured using blood, urine or faeces, and they are often used to study and predict the clinical responses of a subject to the administration of a drug but also the administration of or other products, such as probiotic strains.

The bacterial strain of the genus Bifidobacterium or a mixture of two or more strains thereof may be used in any form capable of exerting the effects described herein. For example, the bacteria may be viable, dormant, inactivated or dead bacteria. Preferably, the bacteria are viable bacteria.

The bacterial strain or strains may comprise whole bacteria or may comprise bacterial components. Examples of such components include bacterial cell wall components such as peptidoglycan, bacterial nucleic acids such as DNA and RNA, bacterial membrane components, and bacterial structural components such as proteins, carbohydrates, lipids and combinations of these such as lipoproteins, glycolipids and glycoproteins.

The bacterial strain or strains may also or alternatively comprise bacterial metabolites. In the present specification the term “bacterial metabolites” includes all molecules produced or modified by the (probiotic) bacteria as a result of bacterial metabolism during growth, survival, persistence, transit or existence of bacteria during the manufacture of the probiotic product and storage and during gastrointestinal transit in a mammal. Examples include all organic acids, inorganic acids, bases, proteins and peptides, enzymes and co-enzymes, amino acids and nucleic acids, carbohydrates, lipids, glycoproteins, lipoproteins, glycolipids, vitamins, all bioactive compounds, metabolites containing an inorganic component, and all small molecules, for example nitrous molecules or molecules containing a sulphurous acid.

Preferably the bacteria comprise whole bacteria, more preferably whole viable bacteria.

Preferably, the Bifidobacterium or Bifidobacteria used in accordance with the present invention is one which is suitable for human and/or animal consumption. A skilled person will be readily aware of specific species and or strains of Bifidobacteria from within the genera described herein which are used in the food and/or agricultural industries and which are generally considered suitable for human and/or animal consumption.

In the present invention, the Bifidobacteria used may be of the same species or may comprise a mixture of species and/or strains.

Suitable bacteria are selected from the species Bifidobacterium lactis, Bifidobacterium bifidium, Bifidobacterium longum, Bifidobacterium animalis, Bifidobacterium breve, Bifidobacterium infantis, Bifidobacterium catenulatum, Bifidobacterium pseudocatenulatum, Bifidobacterium adolescentis, and Bifidobacterium angulatum, and combinations of any thereof.

Preferably, the Bifidobacterium used in the present invention is of the species Bifidobacterium animalis. More preferably, the Bifidobacterium used in the present invention is of the Bifidobacterium animalis ssp. lactis.

In a particularly preferred embodiment, the bacteria used in the present invention are Bifidobacterium animalis ssp. lactis (strain) 420 (B420).

In one embodiment, the bacterium used in the present invention is a probiotic bacterium. In this specification the term ‘probiotic bacterium’ is defined as covering any non-pathogenic bacterium which, when administered live in adequate amounts, confer a health benefit on the host. These probiotic strains generally have the ability to survive the passage through the upper part of the digestive tract. They are non-pathogenic, non-toxic and exercise their beneficial effect on health on the one hand via ecological interactions with the resident flora in the digestive tract, and on the other hand via their ability to influence the immune system in a positive manner via the “GALT” (gut-associated lymphoid tissue). Depending on the definition of probiotics, these bacteria, when given in a sufficient number, have the ability to progress live through the intestine, however they do not cross the intestinal barrier and their primary effects are therefore induced in the lumen and/or the wall of the gastrointestinal tract. They then form part of the resident flora during the administration period. This colonization (or transient colonization) allows the probiotic bacteria to exercise a beneficial effect, such as the repression of potentially pathogenic micro-organisms present in the flora and interactions with the immune system of the intestine.

In a preferred embodiment, the bacterial strain of the genus Bifidobacterium or a mixture thereof is a probiotic bacterial strain.

In another embodiment according to the present invention, the beneficial clinical response to said bacterial strain of the genus Bifidobacterium or a mixture of two or more said strains thereof is at least one of weight management, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in said subject.

In a particular embodiment, the beneficial clinical response to said bacterial strain of the genus Bifidobacterium or a mixture of two or more said strains thereof is at least one of lowering BMI, lowering blood glucose level, reducing body fat, controlling weight gain, inducing weight loss, lowering body fat mass, lowering mesenteric fat mass, lowering trunk fat mass and/or decreasing android fat mass.

In a particular embodiment of the present invention, the biological sample is a blood sample when the biomarker is selected from the group consisting of hPAI1 Total, a bile acid selected from GLCA, LCA, Iso-LCA, and DCA.

In another particular embodiment of the present invention, the biological sample is a faecal sample when the biomarker is a bacteria selected from the group consisting of Coprococcus, Ruminococcus, Akkermansia, Aminipila butyrica, Clostridiales-Incertae Sedis XIII, and Eubacterium coprostanoligenes.

In another particular embodiment of the present invention, the biological sample is a faecal sample when the biomarker is when the biomarker is selected from Pimelic acid and Azelaic acid.

In another particular embodiment of the present invention, the biological sample is a blood sample, when the biomarker is a phospholipid selected from phosphatidylcholine(36:4), phosphatidylcholine(32:1), phosphatidylethanolamine(38:4), phosphatidylethanolamine(36:4), and phosphatidylinositol(40:4).

In an embodiment according to the present invention, the measured level of hPAI1 Total in the biological sample obtained from the subject is above 48245 pg./ml.

In another embodiment, the measured level of GLCA in the biological sample obtained from the subject is above 0.0070 μmol/l.

In another embodiment, the measured level of LCA in the biological sample obtained from the subject is above 0.0380 μmol/l.

In another embodiment, the measured level of relative abundance of Coprococcus in the biological sample obtained from the subject is above 0.0426.

In another embodiment, the measured level of relative abundance of Ruminococcus in the biological sample obtained from the subject is above 0.0631.

In another embodiment, the measured level of relative abundance of Akkermansia in the biological sample obtained from the subject is above 0.0062.

In another embodiment, the measured level of relative abundance Aminipila butyrica in the biological sample obtained from said subject is above 0.00043.

In another embodiment, the measured level of relative abundance Unclassified Clostridiales-Incertae Sedis XIII in the biological sample obtained from said subject is above 0,00018.

In another embodiment, the measured level of relative abundance Eubacterium coprostanoligenes in the biological sample obtained from said subject is above 0,0084.

In another embodiment, the measured level of Iso-LCA in the biological sample obtained from said subject is above 29 μmol/l.

In another embodiment, the measured level of DCA in the biological sample obtained from said subject is above 0.41 nmol/ml.

In another embodiment, the measured level of the phospholipid phosphatidylcholine(36:4) in the biological sample obtained from said subject measured as relative amount calculated as peak area for the phospholipid divided with the total peak area of all identified lipids in positive ionization mode as described herein is above 0.064.

In another embodiment, the measured level of the phospholipid phosphatidylcholine(32:1) in the biological sample obtained from said subject measured as relative amount calculated as peak area for the phospholipid divided with the total peak area of all identified lipids in positive ionization mode as described herein is above 0.0037.

In another embodiment, the measured level of the phospholipid phosphatidylethanolamine(38:4) in the biological sample obtained from said subject measured as relative amount calculated as peak area for the phospholipid divided with the total peak area of all identified lipids in positive ionization mode as described herein is above 0.00074.

In another embodiment, the measured level of the phospholipid phosphatidylethanolamine(36:4) in the biological sample obtained from said subject measured as relative amount calculated as peak area for the phospholipid divided with the total peak area of all identified lipids in negative ionization mode as described herein is above 0.00091.

In another embodiment, the measured level of the phospholipid phosphatidylinositol(40:4) in the biological sample obtained from said subject measured as relative amount calculated as peak area for the phospholipid divided with the total peak area of all identified lipids in negative ionization mode as described herein is above 0.00019.

In another embodiment, the measured level of the pimelic acid in the biological sample obtained from said subject measured as relative amount calculated as peak area of ion 125 m/z divided with the total peak area of all identified peaks as described herein is above 0.000126.

In another embodiment, the measured level of the azelaic acid in the biological sample obtained from said subject measured as relative amount calculated as peak area of ion 83 m/z divided with the total peak area of all identified peaks as described herein is above 0.000135.

In another embodiment, the daily activity of the subject in steps is above 7000, or corresponding physical activity.

In another embodiment, the measured level of the pimelic acid in the biological sample obtained from said subject is above 0.000126.

In another embodiment, the measured level of the azelaic acid in the biological sample obtained from said subject is above 0.000135.

According to the present invention, different analytical techniques may be used to analyse the biological samples according to the present invention. One of the techniques is Liquid chromatography-mass spectrometry (LC-MS), which combines the physical separation capabilities of liquid chromatography (or HPLC) with the mass analysis capabilities of mass spectrometry (MS). Another technique is Gas chromatography-mass spectrometry (GC-MS), which is an analytical method that combines the features of gas-chromatography and mass spectrometry to identify different substances within a sample. A further technique is Nuclear Magnetic Resonance (NMR). These techniques are all well known by a skilled person in the art.

According to a particular embodiment of the present invention, if the biological sample to be analysed is a blood sample, the level of the biomarkers is measured by LC-MS.

According to another particular embodiment, if the biological sample to be analysed is a faecal sample, the level of the biomarkers is measured by LC-MS.

In one embodiment, the measurement of the level of at least one of the biomarkers takes place before administration of the bacterial strain of the genus Bifidobacterium or a mixture of two or more strains thereof to the subject.

In a particular embodiment, the subject according to the present invention is overweight or obese.

In another particular embodiment, the subject according to the present invention has a BMI above 25.

In a particular embodiment, the subject according to the present invention has the hPAI1 Total and the bile acid GLCA higher than the threshold value.

### Dosaae

The Bifidobacterium, such as a strain of Bifidobacterium animalis ssp. lactis, for example Bifidobacterium animalis ssp. lactis (strain) 420 (B420), used in accordance with the present invention may comprise from 10⁶ to 10¹² CFU of bacteria/g of support, and more particularly from 10⁸ to 10¹² CFU of bacteria/g of support, preferably 10⁹ to 10¹² CFU/g for the lyophilized form.

Suitably, the Bifidobacterium, such as a strain of Bifidobacterium animalis ssp. lactis, for example Bifidobacterium animalis ssp. lactis (strain) 420 (B420), may be administered at a dosage of from about 10⁶ to about 10¹² CFU of microorganism/dose, preferably about 10⁸ to about 10¹² CFU of microorganism/dose. By the term “per dose” it is meant that this amount of microorganism is provided to a subject either per day or per intake, preferably per day. For example, if the microorganism is to be administered in a food product, for example in a yoghurt, then the yoghurt will preferably contain from about 10⁸ to 10¹² CFU of the microorganism. Alternatively, however, this amount of microorganism may be split into multiple administrations each consisting of a smaller amount of microbial loading—so long as the overall amount of microorganism received by the subject in any specific time, for instance each 24-hour period, is from about 10⁶ to about 10¹² CFU of microorganism, preferably 10⁸ to about 10¹² CFU of microorganism.

In accordance with the present invention an effective amount of at least one strain of a microorganism may be at least 10⁶ CFU of microorganism/dose, preferably from about 10⁶ to about 10¹² CFU of microorganism/dose, preferably about 10⁸ to about 10¹² CFU of microorganism/dose.

In one embodiment, preferably the Bifidobacterium, such as a strain of Bifidobacterium animalis ssp. lactis, for example Bifidobacterium animalis ssp. lactis (strain) 420 (B420), may be administered at a dosage of from about 10⁶ to about 10¹² CFU of microorganism/day, preferably about 10⁸ to about 10¹² CFU of microorganism/day. Hence, the effective amount in this embodiment may be from about 10⁶ to about 10¹² CFU of microorganism/day, preferably about 10⁸ to about 10¹² CFU of microorganism/day.

CFU stands for “colony-forming units”. By ‘support’ is meant the food product, dietary supplement or the pharmaceutically acceptable formulation.

### Effects/Subjects/Medical Indications

The bacterial strain of the genus Bifidobacterium to which the present invention relates is administered to a subject, including for example livestock (including cattle, horses, pigs and sheep), and humans. In some aspects of the present invention the subject is a companion animal (including pets), such as a dog or a cat for instance. In some aspects of the present invention, the subject may suitably be a human.

The bacterial strain or strains according to the present invention may have a beneficial clinical response for at least one of weight management, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in the subject.

The bacterial strain or strains to which the present invention relates may have a beneficial clinical response for at least one of lowering BMI, lowering blood glucose level, reducing body fat, controlling weight gain, inducing weight loss, lowering body fat mass, lowering mesenteric fat mass, lowering trunk fat mass and/or decreasing android fat mass, in the subject.

### Compositions

While it is possible to administer the bacterial strain or strains alone, in a particular embodiment of the present invention the bacterial strain or strains are used in combination with one or more fibres and/or prebiotics. In another particular embodiment, the fibres and/or the prebiotic is polydextrose.

In a particular embodiment, the bacterial strain (or strains) is in the form of a composition.

While is it possible to administer the composition alone according to the present invention (i.e. without any support, diluent or excipient), the composition is typically and preferably administered on or in a support as part of a product, in particular as a component of a food product, a dietary supplement or a pharmaceutical composition or formulation. These products typically contain additional components well known to those skilled in the art.

By “composition” it is understood the combination of 2 or more substances. The substances may be chemical substances or biological substances, such as bacteria, including the substance that has the desired effect.

By “formulation”, it is understood the process or composition in which different chemical and/or biological substances, including the substance having the desired effect, are combined to produce a final product. Composition and formulation may be used interchangeably.

Any product which can benefit from the composition may be used in the present invention. These include but are not limited to foods, particularly fruit conserves and dairy foods and dairy food-derived products, and pharmaceutical products.

When used as, or in the preparation of, a food, such as functional food, the composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the composition of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, acidified soy/juice beverage, aseptic/retorted chocolate drink, bar mixes, beverage powders, calcium fortified soy/plain and chocolate milk, calcium fortified coffee beverage.

The composition can further be used as an ingredient in food products such as American cheese sauce, anti-caking agent for grated & shredded cheese, chip dip, cream cheese, dry blended whip topping fat free sour cream, freeze/thaw dairy whipping cream, freeze/thaw stable whipped topping, low fat and light natural cheddar cheese, low fat Swiss style yoghurt, aerated frozen desserts, hard pack ice cream, label friendly, improved economics & indulgence of hard pack ice cream, low fat ice cream: soft serve, barbecue sauce, cheese dip sauce, cottage cheese dressing, dry mix Alfredo sauce, mix cheese sauce, dry mix tomato sauce and others.

The composition of the present invention may be used as a food ingredient and/or feed ingredient.

As used herein the term “food ingredient” or “feed ingredient” includes a formulation which is or can be added to functional foods or foodstuffs as a nutritional supplement.

The food ingredient may be in the form of a solution or as a solid, depending on the use and/or the mode of application and/or the mode of administration.

### Food Products

In one embodiment, the bacterial strain (or strains) according to the present invention is in the form of a food product, such as a food supplement, a drink or a powder based on milk. Here, the term “food” is used in a broad sense and covers food for humans as well as food for animals (i.e. a feed). In a preferred aspect, the food is for human consumption.

The food may be in the form of a solution or as a solid, depending on the use and/or the mode of application and/or the mode of administration.

Advantageously, where the product is a food product, comprising the bacterium of the genus Bifidobacterium or a mixture thereof, and one or more prebiotics and/or fibres, it should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment.

### Food Supplements

The bacterial strain (or strains) of the present invention may be—or may be added to—dietary supplements, also referred to herein as food supplements.

Here, the term “dietary supplement” is a product intended for ingestion that contains a “dietary ingredient” intended to add further nutritional value to (supplement) the diet. A “dietary ingredient” may be one, or any combination, of the following substances: a vitamin, a mineral, an herb or other botanical, an amino acid, a dietary substance for use by people to supplement the diet by increasing the total dietary intake, a concentrate, metabolite, constituent, or extract.

Dietary supplements may be found in many forms such as tablets, capsules, soft gels, gel caps, liquids, or powders. Some dietary supplements can help ensure that you get an adequate dietary intake of essential nutrients; others may help you reduce your risk of disease.

### Functional Foods

The bacterial strain (or strains) of the present invention may be—or may be added to—functional foods.

As used herein, the term “functional food” means food which is capable of providing, not only a nutritional effect, but is also capable of delivering a further beneficial effect to consumer.

Accordingly, functional foods are ordinary foods that have components or ingredients (such as those described herein) incorporated into them that impart to the food a specific functional—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction but is also capable of delivering a therapeutic (or other beneficial) effect to the consumer. Nutraceuticals cross the traditional dividing lines between foods and medicine.

### Medical Food

In one embodiment, the bacterial strain (strains) of the present invention, for example Bifidobacterium animalis ssp. lactis (strain) 420 (B420), is in the form of a medical food product.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Pharmaceutical

The bacterial strain (or strains) of the present invention may be used as—or in the preparation of—a pharmaceutical formulation or composition. Here, the term “pharmaceutical” is used in a broad sense—and covers pharmaceuticals for humans as well as pharmaceuticals for animals (i.e. veterinary applications). In a preferred aspect, the pharmaceutical is for human use and/or for animal husbandry.

The pharmaceutical can be for therapeutic purposes—which may be curative or palliative or preventative in nature.

A pharmaceutically acceptable formulation or support or composition may be for example a formulation or support in the form of compressed tablets, tablets, capsules, ointments, suppositories or drinkable solutions. Other suitable forms are provided below.

When used as—or in the preparation of—a pharmaceutical, the bacterial strain (or strains) of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a solution or as a solid—depending on the use and/or the mode of application and/or the mode of administration.

The bacterial strain (or strains) of the present invention may be used as pharmaceutical ingredients. Here, the bacterial strain (or strains) may be the sole active component, or it may be at least one of a number (i.e. 2 or more) of active components.

The pharmaceutical formulations may be used according to the present invention in the form of solid or liquid preparations or alternatives thereof. Examples of solid preparations include, but are not limited to tablets, capsules, dusts, granules and powders which may be wettable, spray-dried or freeze-dried. Examples of liquid preparations include, but are not limited to, aqueous, organic or aqueous-organic solutions, suspensions and emulsions.

Suitable examples of forms include one or more of: tablets, pills, capsules, ovules, solutions or suspensions, which may contain flavouring or colouring agents, for immediate-, delayed-, modified-, sustained-, pulsed- or controlled-release applications.

By way of example, if the pharmaceutical formulation of the present invention is used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Further examples of form include creams. For some aspects the microorganism used in the present invention may be used in pharmaceutical and/or cosmetic creams such as sun creams and/or after-sun creams for example.

In one aspect, the pharmaceutical formulation according to the present invention may be administered in an aerosol, for example by way of a nasal spray, for instance for administration to the respiratory tract.

### Medicament

In one embodiment, the pharmaceutical acceptable formulation of the present invention is a medicament.

The term “medicament” as used herein encompasses medicaments for both human and animal usage in human and veterinary medicine. In addition, the term “medicament” as used herein means any substance which provides a therapeutic and/or beneficial effect. The term “medicament” as used herein is not necessarily limited to substances which need Marketing Approval, but may include substances which can be used in cosmetics, nutraceuticals, food (including feeds and beverages for example), probiotic cultures, and natural remedies. In addition, the term “medicament” as used herein encompasses a product designed for incorporation in animal feed, for example livestock feed and/or pet food.

### Prebiotics

In one embodiment, the bacterial strain (or strains) according to the present invention is used in combination with one or more fibres and/or prebiotics.

Prebiotics are a category of functional food, defined as non-digestible food ingredients that beneficially affect the host by selectively stimulating the growth and/or activity of one or a limited number of bacteria (particularly, although not exclusively, probiotics, Bifidobacteria and/or lactic acid bacteria) in the colon, and thus improve host health. Typically, prebiotics are carbohydrates (such as oligosaccharides), but the definition does not preclude non-carbohydrates. The most prevalent forms of prebiotics are nutritionally classed as soluble fibres. To some extent, many forms of dietary fibres exhibit some level of prebiotic effect.

In one embodiment, a prebiotic is a selectively fermented ingredient that allows specific changes, both in the composition and/or activity in the gastrointestinal microflora that confers benefits upon host well-being and health.

Suitably, the prebiotic may be used according to the present invention in an amount of 0.01 to 100 g/day, preferably 0.1 to 50 g/day, more preferably 0.5 to 20 g/day. In one embodiment, the prebiotic may be used according to the present invention in an amount of 1 to 10 g/day, preferably 2 to 9 g/day, more preferably 3 to 8 g/day. In another embodiment, the prebiotic may be used according to the present invention in an amount of 5 to 50 g/day, preferably 10 to 25 g/day.

Examples of dietary sources of prebiotics include soybeans, inulin sources (such as Jerusalem artichoke, jicama, and chicory root), raw oats, unrefined wheat, unrefined barley and yacon.

Examples of suitable prebiotics include alginate, xanthan, pectin, locust bean gum (LBG), inulin, guar gum, galacto-oligosaccharide (GOS), fructo-oligosaccharide (FOS), polydextrose (i.e. Litesse®), lactitol, lactosucrose, soybean oligosaccharides, isomaltulose (Palatinose™), isomalto-oligosaccharides, gluco-oligosaccharides, xylooligosaccharides, manno-oligosaccharides, beta-glucans, cellobiose, raffinose, gentiobiose, melibiose, xylobiose, cyciodextrins, isomaltose, trehalose, stachyose, panose, pullulan, verbascose, galactomannans, and all forms of resistant starches.

A particularly preferred example of a fibre and/or prebiotic is polydextrose.

The combination of Bifidobacterium and one or more fibres and/or prebiotics according to the present invention exhibits a synergistic effect (i.e. an effect which is greater than the additive effect of the bacteria when used separately). Without wishing to be bound by theory, it is believed that such a combination is capable of selectively stimulating the growth and/or activity of the Bifidobacteria in the colon, and thus improving its effect and the host health.

In one embodiment, the bacterial strain or strains of the genus Bifidobacterium used in the combination with one or more fibres and/or prebiotics is of the species Bifidobacterium animalis. More preferably, the Bifidobacterium used in the combination with one or more fibres and/or prebiotics is of the Bifidobacterium animalis ssp. lactis.

In a particularly preferred embodiment, the Bifidobacterium used in the combination with one or more fibres and/or prebiotics is the Bifidobacterium animalis ssp. lactis (strain) 420 (B420).

Another particular preferred embodiment of a fibre and/or prebiotic is Litesse© Ultra polydextrose (LU).

Litesse® Ultra polydextrose (LU), is a randomly cross-linked polymer of glucose, which remains undigested by the host and may increase the number of Bifidobacteria in a colonic continuous culture system.

### NUMBERED EMBODIMENTS OF THE INVENTION

1. A method of identifying a subject having an increased probability of having a beneficial clinical response to administration of a bacterial strain of the genus Bifidobacterium or a mixture of two or more strains thereof, the method comprising the steps of,
   i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps or corresponding physical activity; and
   ii. comparing said level to a threshold value,
      wherein when the measured level of at least one of the biomarkers is higher than said threshold value, the subject is identified as having said increased probability.
2. The method according to embodiment 1, wherein the beneficial clinical response to said bacterial strain of the genus Bifidobacterium or a mixture of two or more said strains thereof is at least one of weight management, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in said subject.
3. The method according to embodiment 2, wherein the beneficial clinical response to said bacterial strain of the genus Bifidobacterium or a mixture of two or more said strains thereof is at least one of lowering BMI, reducing body fat, lowering blood glucose level, controlling weight gain, inducing weight loss, lowering body fat mass, lowering mesenteric fat mass, lowering trunk fat mass and/or decreasing android fat mass, in said subject.
4. The method according to any one of the preceding embodiments, wherein said biological sample is a blood sample, when the biomarker is selected from the group consisting of hPAI1 Total, a bile acid selected from GLCA, LCA, Iso-LCA, and DCA.
5. The method according to any one of embodiments 1-4, wherein said biological sample is a fecal sample, when the biomarker is a bacteria selected from the group consisting of Coprococcus, Ruminococcus, Akkermansia, Aminipila butyrica, Clostridiales-Incertae Sedis XIII, and Eubacterium coprostanoligenes.
6. The method according to any one of embodiments 1-5, wherein said biological sample is a fecal sample when the biomarker is selected from Pimelic acid and Azelaic acid.
7. The method according to any one of the preceding embodiments, wherein said biological sample is a blood sample, when the biomarker is a phospholipid selected from phosphatidylcholine(36:4), phosphatidylcholine(32:1), phosphatidylethanolamine(38:4), phosphatidylethanolamine(36:4), and phosphatidylinositol(40:4).
8. The method according to any one of the preceding embodiments, wherein the bacterial strain of the genus Bifidobacterium or a mixture thereof is a probiotic bacterial strain.
9. The method according to any one of the preceding embodiments, wherein said bacterial strain of the genus Bifidobacterium or a mixture of two or more said strains thereof if of the species Bifidobacterium animalis, preferably of the species Bifidobacterium animalis subsp. lactis.
10. The method according to embodiment 9, wherein the bacterial strain of the species Bifidobacterium animalis subsp. Lactis is strain B420.
11. The method according to any one of the preceding embodiments, wherein the measured level of hPAI1 Total in the biological sample obtained from said subject is above 48245 μg/ml.
12. The method according to any one of the embodiments 1-11, wherein the measured level of GLCA in the biological sample obtained from said subject is above 0.0070 μmol/l.
13. The method according to any one of the embodiments 1-12, wherein the measured level of LCA in the biological sample obtained from said subject is above 0.0380 μmol/l.
14. The method according to any one of the embodiments 1-13, wherein the measured level of relative abundance of Coprococcus in the biological sample obtained from said subject is above 0.0426.
15. The method according to any one of the embodiments 1-14, wherein the measured level of relative abundance of Ruminococcus in the biological sample obtained from said subject is above 0.0631.
16. The method according to any one of the embodiments 1-15, wherein the measured level of relative abundance of Akkermansia in the biological sample obtained from said subject is above 0.0062.
17. The method according to any one of the embodiments 1-16, wherein the measured level of relative abundance Aminipila butyrica in the biological sample obtained from said subject is above 0.00043.
18. The method according to any one of the embodiments 1-17, wherein the measured level of relative abundance Unclassified Clostridiales-Incertae Sedis XIII in the biological sample obtained from said subject is above 0.00018.
19. The method according to any one of the embodiments 1-18, wherein the measured level of relative abundance Eubacterium coprostanoligenes in the biological sample obtained from said subject is above 0.0084.
20. The method according to any one of the embodiments 1-19, wherein the measured level of Iso-LCA in the biological sample, such as in feces, obtained from said subject is above 29 μmol/l.
21. The method according to any one of the embodiments 1-20, wherein the measured level of DCA in the biological sample, such as in blood serum, obtained from said subject is above 0.41 nmol/ml.
22. The method according to any one of the embodiments 1-21, wherein the measured level of the phospholipid phosphatidylcholine(36:4) in the biological sample obtained from said subject measured as relative amount calculated as peak area for the phospholipid divided with the total peak area of all identified lipids in positive ionization mode as described herein is above 0.064.
23. The method according to any one of the embodiments 1-22, wherein the measured level of the phospholipid phosphatidylcholine(32:1) in the biological sample obtained from said subject measured as relative amount calculated as peak area for the phospholipid divided with the total peak area of all identified lipids in positive ionization mode as described herein is above 0.0037.
24. The method according to any one of the embodiments 1-23, wherein the measured level of the phospholipid phosphatidylethanolamine(38:4) in the biological sample obtained from said subject measured as relative amount calculated as peak area for the phospholipid divided with the total peak area of all identified lipids in positive ionization mode as described herein is above 0.00074.
25. The method according to any one of the embodiments 1-24, wherein the measured level of the phospholipid phosphatidylethanolamine(36:4) in the biological sample obtained from said subject measured as relative amount calculated as peak area for the phospholipid divided with the total peak area of all identified lipids in negative ionization mode as described herein is above 0.00091.
26. The method according to any one of the embodiments 1-25, wherein the measured level of the phospholipid phosphatidylinositol(40:4) in the biological sample obtained from said subject measured as relative amount calculated as peak area for the phospholipid divided with the total peak area of all identified lipids in negative ionization mode as described herein is above 0.00019.
27. The method according to any one of the embodiments 1-26, wherein the measured level of the pimelic acid in the biological sample obtained from said subject measured as relative amount calculated as peak area of ion 125 m/z divided with the total peak area of all identified peaks as described herein is above 0.000126.
28. The method according to any one of the embodiments 1-27, wherein the measured level of the azelaic acid in the biological sample obtained from said subject measured as relative amount calculated as peak area of ion 83 m/z divided with the total peak area of all identified peaks as described herein is above 0.000135.
29. The method according to any one of the preceding embodiments, wherein the measured level when the biological sample is a blood sample is measured by LC-MS.
30. The method according to any one of the preceding embodiments, wherein the measured level when the biological sample is a fecal sample is measured by GC-MS and/or NMR.
31. The method according to any one of the preceding embodiments, wherein the measurement of the level of at least one of the biomarkers takes place before administration of the bacterial strain of the genus Bifidobacterium or a mixture of two or more strains thereof to said subject.
32. The method according to any one of the preceding embodiments, wherein the subject is overweight or obese.
33. The method according to any one of the preceding embodiments, wherein the subject has a BMI above 25.
34. The method according to any one of the preceding embodiments, wherein said subject has the hPAI1 Total and the bile acid GLCA higher than said threshold value.
35. The method according to any one of the preceding embodiments, wherein the bacterial strain is used in combination with one or more fibres and/or prebiotics.
36. The method according to any one of the preceding embodiments, wherein the fibres and/or the prebiotic is polydextrose.
37. The method according to any one of the preceding embodiments, wherein the bacterial strain is in the form of a composition, a food product, a dietary supplement or a pharmaceutically acceptable formulation.
38. The method according to embodiment 37, wherein the pharmaceutically acceptable formulation is a medicament.
39. The method according to embodiment 37, wherein the food product is a medical food product.
40. The method according to any one of the preceding embodiments, wherein the daily activity of the subject in steps is above 7000, or corresponding physical activity.
41. Use of a bacterial strain of the genus Bifidobacterium or a mixture of two or more strains thereof for at least one of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject, wherein said subject has been identified as having an increased probability of having a beneficial clinical response to administration of said bacterial strain of the genus Bifidobacterium or a mixture of two or more strains thereof, by,
   i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps; and
   ii. comparing said level to a threshold value,
      wherein when the measured level of at least one of the biomarkers is higher than said threshold value, the subject is identified as having said increased probability.
42. The use of a bacterial strain of the genus Bifidobacterium or a mixture of two or more strains according to embodiment 39 as further defined in any one of the embodiments 1-40.
43. Bacterial strain of the genus Bifidobacterium or a mixture of two or more strains thereof for use in managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject, wherein said subject has been identified as having an increased probability of having a beneficial clinical response to administration of said bacterial strain of the genus Bifidobacterium or a mixture of two or more strains thereof, by,
   i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps; and
   ii. comparing said level to a threshold value,
      wherein when the measured level of at least one of the biomarkers is higher than said threshold value, the subject is identified as having said increased probability.
44. The bacterial strain of the genus Bifidobacterium or a mixture of two or more strains for use according to embodiment 43 as further defined in any one of the embodiments 1-40.
45. Method of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject in need thereof, said method comprising administering a bacterial strain of the genus Bifidobacterium or a mixture of two or more strains thereof to said subject, wherein said subject has been identified as having an increased probability of having a beneficial clinical response to the administration of said bacterial strain of the genus Bifidobacterium or a mixture of two or more strains thereof, by,
   i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps; and
   ii. comparing said level to a threshold value,
      wherein when the measured level of at least one of the biomarkers is higher than said threshold value, the subject is identified as having said increased probability.
46. The method according to embodiment 45 as further defined in any one of the embodiments 1-40.
47. A method of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject in need thereof, said method comprising
   i. Prescribing to the subject a daily personal activity of more than 7000 steps, or corresponding physical activity; and
   ii. administering a bacterial strain of the genus Bifidobacterium or a mixture of two or more strains thereof to the subject having obtained the activity under i), which subject has an increased probability of having a beneficial clinical response to the administration of said bacterial strain of the genus Bifidobacterium or a mixture of two or more strains thereof.
48. The method according to embodiment 47, which bacterial strain of the genus Bifidobacterium or a mixture of two or more strains thereof is defined in any one of the embodiments 8-10.

### EXAMPLES

### Introduction

The following examples are based on a multivariate data analysis of the results from the MetSProb study (Stenman 2016). MetSProb is a randomised, double-blind, placebo controlled clinical study (ClinicalTrials.gov NCT01978691) conducted in Finland for overweight and obese study population. The results have been published in two original papers in 2016 (Stenman et al 2016) and 2019 (Hibberd et al. 2019).

Stenman et al reported the primary results showing that a probiotic alone or together with a prebiotic controls body fat mass in healthy overweight or obese subjects. The second publication Hibberd et al. (2019) investigated the associations between the gut microbiota with the observed clinical benefits after the 6-month interventions with prebiotic, probiotic and synbiotic products, showing that consumption of Probiotic strain B420 and its combination with polydextrose (PDX) resulted in alterations of the gut microbiota and its metabolism, indicating a possibility to support improved gut barrier function and obesity-related markers.

A large number of parameters (130 in all, such as glucose, insulin, hPAI1 Total, bile acids, bacterias etc.) were measured during the MetSProb study (Stenman 2016) in blood samples and fecal samples taken from the subjects of the study population.

Applying a multivariate Latent Class Analysis statistical model to all of the measured data for all 130 parameters from the subjects in the MetSProb human clinical study, surprisingly resulted in a set of only six predictive variables at baseline for weight loss or lowered weight gain for the group supplied with B420 over a 6-months intervention compared to the placebo group. Weight loss was measured in the study as Body Mass Index (BMI), DXA Trunk Fat (DXA_TrFa), DXA Android Fat (DXA_AnFa) and DXA Total Fat (DXA_fat).

The six predictor variables are human Plasminogen activator inhibitor-1 (HPAI1 Total), the secondary bile acids Glucolithocholic acid (GLCA) and Lithocholic acid (LCA) measured in the blood as well as faecal bacterial genera Coprococcus, Ruminococcus and Akkermansia.

A statistical MANOVA analysis confirmed the findings of the six predictor variables.

Subjects can be classified as responders or non-responders to the Bifidobacterium B420 intervention from one or more of the six predictor variables. Samples can be withdrawn as blood or as fecal samples and for the prediction a median threshold can be used as illustrated in the examples below.

### Participants and Study Design (Hibberd et al. 2019)

A double-blind, randomised, parallel, placebo-controlled clinical trial was conducted in overweight and obese adults to investigate the effect of probiotic, prebiotic or synbiotic (probiotic+prebiotic) intervention on body fat mass and obesity-related markers. The study populations and clinical outcomes were detailed previously (Stenman et al., 2016) and are summarised here.

Study participants were recruited from four clinical research centres in southern Finland between December 2013 and October 2014. Participants were overweight or obese (body mass index (BMI) 28.0-34.9) but otherwise healthy. Exclusion criteria included diagnosed type 1 or type 2 diabetes or cardiovascular disease, bariatric surgery, pregnant or breastfeeding women, recent consumption of laxatives, immunomodulatory drugs, high-dose vitamin D supplements, fibre supplements (within previous 6 weeks), probiotics (6 weeks), antibiotics (2 months), anti-obesity drugs (3 months), participation in a weight loss program (3 months) or a weight change of 3 kg in the previous 3 months. Participants were randomised into four groups: (1) placebo, 12 g/day of microcrystalline cellulose; (2) prebiotic LU, 12 g/day; (3) probiotic Bifidobacterium animalis subsp. lactis 420™ (B420), 1010 cfu/day in 12 g microcrystalline cellulose; (4) synbiotic LU+B420, 1010 cfu/day of B420 in 12 g/day LU. The study products were manufactured by DuPont Nutrition and Health (Madison, WI, USA) as sachets that participants were instructed to mix into a commercially available fruit smoothie (130 kcal) once daily while maintaining their regular diet and exercise habits. Participants attended clinic visits for screening and baseline assessments, and subsequently after 2 months, 4 months and 6 months of study product usage. A follow-up visit occurred one month after the completion of the intervention. Participants were monitored for study product compliance and protocol violations as described previously (Stenman et al., 2016), and the protocol compliant population (n=134) baseline characteristics are summarised in Table 1. Body composition and metabolic biomarkers that were measured from participants included dual-energy X-ray absorptiometry (DXA) measured body fat mass and lean body mass (total, android (waist area), gynoid (hip area), trunk, legs, arms), BMI, waist and hip circumference as well as blood markers in fasted participants for serum hsCRP, serum glucose, serum insulin, blood glycosylated haemoglobin, serum lipids (total cholesterol, low-density lipoprotein (LDL), high-density lipoprotein and triglycerides), serum cortisol, and serum liver markers (aspartate aminotransferase; alanine aminotransferase; and gamma-glutamyl transferase) (Stenman et al., 2016).

### Sample Collection and Processing (Hibberd et al. 2019).

Faecal samples were obtained from participants at the baseline visit, during study intervention visits (2, 4 and 6 months) and one month after end of treatment (+1 month). Samples were frozen immediately and stored at −80° C. Until analysis. The faecal microbiota was analysed from all samples, and faecal metabolites and bile acids were analysed from baseline and 6-month visits. Fasting blood samples collected at baseline and 6-month visits were used for analysis of plasma bile acids.

### Example 1: Statistical Analyses

This study aims at identifying baseline biomarkers that are associated to responder status during the intervention period of individual subjects receiving an active treatment (B420) compared to a Placebo treatment.

### Experiment

The MetSProb human clinical study is a cross-section design, where subjects are receiving either a Placebo (N=36) treatment or an active (B420, N=25) treatment and multiple parameters are evaluated at 2 paired timepoints (v2=baseline, v8=after 6 months of intervention).

The objective is to explore if baseline (v2) biomarkers are associated to during intervention period (v8-v2) responder status for strain B420 vs Placebo.

Responder status is based on 4 weight and body composition parameters (Body Mass Index (BMI), DXA Trunk Fat (DXA_TrFa), DXA Android Fat (DXA_AnFa), DXA Total Fat (DXA_fat)) and responders are those subjects experiencing a weight loss (i.e. the v8-v2 difference is <0). Otherwise, subjects are classified as non-responders.

Plotting, an arbitrary weight and body composition parameter for v8-v2 against an arbitrary baseline (v2) parameter for the 2 treatment populations (B420 and Placebo), the pattern of difference in slopes for B420 vs Placebo will show if it's a biomarker or not. If the difference (B420 vs Placebo) in slopes is significantly different from zero at level 5%, it's a biomarker and if the sign of the difference is negative (positive) it means that high (low) values of the biomarker increases the probability of being a responder.

The software used is SAS 9.4 (SAS Institute Inc., Cary, NC, USA. 2016).

### Exploratory Statistical Analysis

A multivariate latent class analysis model is fitted to all parameters at the 2 time points (v2, v8) and from this model is derived a directed sub-model fitting the 4 weight and body composition parameters (BMI, DXA_TrFa, DXA_AnFa, DXA_fat)(v8-v2) vs all baseline (v2) parameters. This allows a statistical quantification of the difference (B420 vs Placebo) in slopes (denoted “j1”).

The result from the analysis is outlined in Table 2.

TABLE 2
Summary of latent class analysis results.

| | Latent Class Analysis - Z-scores | | |
|---|---|---|---|
| | | Non- | |
| | Responders | Responders | Predictive |
| Predictor variables | (R_j1) | (NR_j1) | (j1) |
| hPAI1 Total (pg/mL) | −2.55 | −4.52 | −4.99 |
| Glucolithocholic acid (GLCA, | −2.91 | −3.03 | −4.20 |
| μmol/L) | | | |
| Lithocholic acid (LCA, μmol/L) | −2.65 | −2.29 | −3.49 |
| Coprococcus (rel. abundance) | −1.53 | −2.22 | −2.66 |
| Ruminococcus (rel. abundance) | −1.06 | −2.35 | −2.41 |
| Akkermansia (rel. abundance) | −0.96 | −2.39 | −2.37 |

Significant Z-score is an absolute value >1.96

The 3 information carriers (j1, R_j1, NR_j1) are the Z-scores for difference (B420 vs Placebo) in slopes for respectively the prospective setup (without knowing the responder status; j1) and the retrospective setup for responders (R_j1) and non-responders (NR_j1).

If “j1” is significant it means that either (R_j1, NR_j1) or both are significant.

If “j1” is significant it means a weight reduction; if “R_j1” is significant it means a weight loss; if “NR_j1” is significant it means a weight gain reduction.

From Table 2 is observed, that 6 parameters (hPAI1 Total, GLCA, LCA, Coprococcus, Ruminococcus, Akkermansia) are identified as baseline biomarkers, all with a negative sign meaning that high values are associated with an increased probability of being a responder.

### Confirmatory Statistical Analysis

The 6 predictor biomarkers identified in Table 2 are all confirmed as genuine biomarkers from a simpler modelling.

Let the responses be the 4 weight and body composition measures (BMI, DXA_TrFa, DXA_AnFa, DXA_fat) for v8-v2 in raw scale (applying mean centring and unit scaling). Let the regressor be a single and arbitrary of the 6 biomarkers for baseline (v2) in raw scale. Let the treatment identifier (B420, Placebo) be a class variable.

Fit a MANOVA (Multivariate Analysis of Variance; type=CS) using PROC MIXED with different slopes for the 2 treatments (1B420 and Placebo) and estimate the average of differences in slope (B420-Placebo). The p-value (Pr<t) is 1-sided (lower).

In Table 3 is the SAS-output (PROC MIXED) for each of the 6 biomarkers separately.

TABLE 3
Results of MANOVA analysis.

| | PROCMIXED Estimates output | | | | | |
|---|---|---|---|---|---|---|
| | Estimates | | | | | |
| | | | Standard | | | |
| _NAME_{—} | Label | Estimate | Error | DF | t Value | Pr < t |
| hPAI 1total | B420-Placebo | −0.00005 | 0.000015 | 225 | −3.55 | 0.0002 |
| LCA | B420-Placebo | −37.3006 | 11.1834 | 201 | −3.34 | 0.0005 |
| GLCA | B420-Placebo | −372.51 | 97.9545 | 201 | −3.8 | <.0001 |
| Coprococcus | B420-Placebo | −73.6344 | 28.2669 | 221 | −2.6 | 0.0049 |
| Ruminococcus | B420-Placebo | −22.4521 | 10.4378 | 221 | −2.15 | 0.0163 |
| Akkermansia | B420-Placebo | −60.8013 | 32.1681 | 221 | −1.89 | 0.03 |

Threshold values for the predictor variables can be calculated from the measured data as the median (50-percentile) and the 33:66-percentile range. The threshold values are depicted in Table 4.

TABLE 4
Summary of the exploratory and confirmatory statistics analyses. The Threshold
value is the median (50-percentile) and the 33:66-percentile range.

| | | | | | Predictive |
|---|---|---|---|---|---|
| | Latent Class Analysis - Z-score | | | MANOVA | Threshold value |
| Predictor | Responders | Non-Responders | Predictive | t-value | Median |
| variables | (R_j1) | (NR_j1) | (j1) | (p-value) | [33:66-percentile] |
| hPAI1 Total | −2.55 | −4.52 | −4.99 | −3.55 | 48245 |
| (pg/mL) | | | | (0.0002) | [40653:53294] |
| Glucolithocholic | −2.91 | −3.03 | −4.20 | −3.80 | 0.0070 |
| acid (GLCA, | | | | (<0.0001) | [0.0033:0.0078] |
| μmol/L) | | | | | |
| Lithocholic acid | −2.65 | −2.29 | −3.49 | −3.34 | 0.0380 |
| (LCA, μmol/L) | | | | (0.0005) | [0.0301:0.0583] |
| Coprococcus | −1.53 | −2.22 | −2.66 | −2.60 | 0.0426 |
| (rel. | | | | (0.0049) | [0.0360:0.0537] |
| abundance) | | | | | |
| Ruminococcus | −1.06 | −2.35 | −2.41 | −2.15 | 0.0631 |
| (rel. | | | | (0.0163) | [0.0319:0.0921] |
| abundance) | | | | | |
| Akkermansia | −0.96 | −2.39 | −2.37 | −1.89 | 0.0062 |
| (rel. | | | | (0.0300) | [0.0041:0.0118] |
| abundance) | | | | | |
| | Significant | | | Significant | |
| | Z-score is | | | p-value <0.05 | |
| | an absolute | | | | |
| | value >1.96 | | | | |

The MANOVA analysis confirms the findings of the latent class analysis.

### Example 2—hPAI1 Total Results

hPAI1 Total is human Plasminogen activator inhibitor-1 (PAI-1) which is a serine protease inhibitor (serpin) encoded by the SERPINE1 gene. Elevated PAI-1 is a risk factor for thrombosis and atherosclerosis, but also other diseases like cancer.

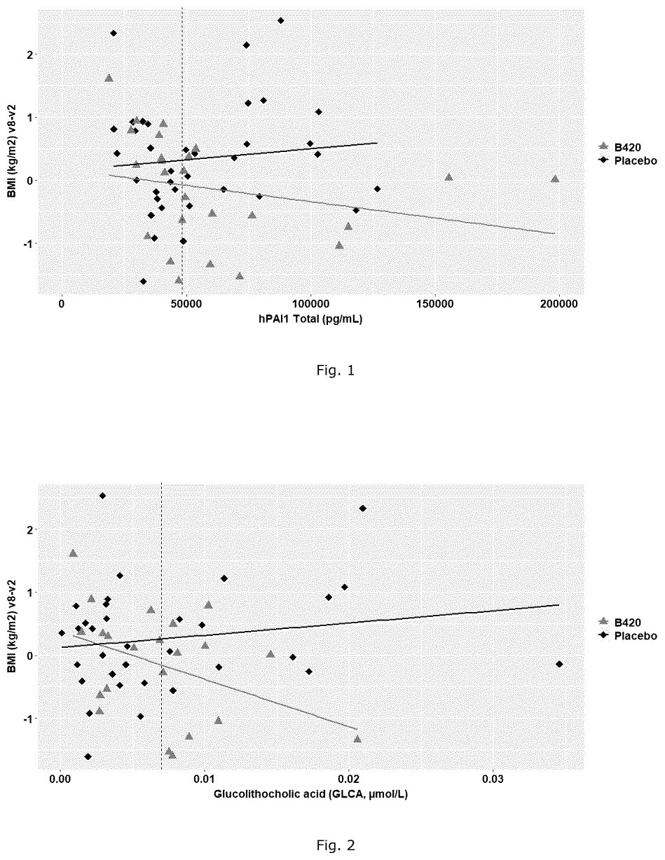

The total human plasminogen activator inhibitor-1 (hPAI1 Total) in blood samples was found to be a significant predictor of weight loss by the Latent Class Analysis model (Example 1). As both the predictive Z-score and the responders Z-score are significant and negative, it means that a high level of hPAI1 Total is predictive of weight loss during a 6 months intervention with B420. The blood level threshold for predictive weight loss of subjects is in the range [40653:53294] pg/mL with a median of 48245 pg/mL. In FIG. 1 is the change in BMI (kg/m²) over the 6-month intervention (v8-v2) plotted against hPAI1 Total at baseline for each subject in the intervention group B420 (N=25) and Placebo (N=36). The median threshold is plotted as well.

From the confirmatory analysis (Table 4), using linear regression lines to illustrate the difference, it can be observed from FIG. 1 that the placebo group is gaining weight/positive increase in BMI, while the B420 group is losing weight/negative trendline for BMI during the 6 months intervention.

### Method Description

Blood clinical determination of total human plasminogen activator inhibitor-1 (hPAI1 Total) is measured using ELISA assay (pg/mL) as described in Stenman 2016—Supplementary Information.

Statistical analysis: see Example 1.

### Example 3—Bile Acid Results

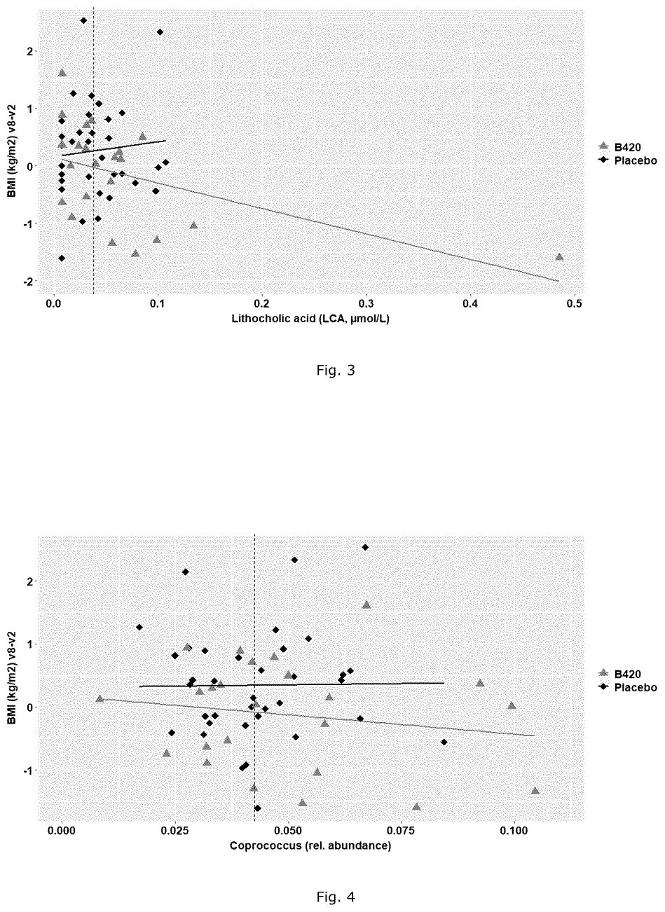

The secondary bile acids Glucolithocholic acid (GLCA) and Lithocholic acid (LCA) in blood samples were found to be significant predictors of weight loss by the Latent Class Analysis model (Example 1). As both the predictive Z-score and Responders Z-score are significant and negative for both GLCA and LCA, it means that a high level of one or the other secondary bile acid is predictive of weight loss during the 6-month intervention with B420. The blood level threshold for predictive weight loss of subjects should be in the ranges [0.0033:0.0078] and [0.0301:0.0583] with median values of 0.0070 μmol/L and 0.0380 μmol/L for GLC and LCA respectively. In FIGS. 2 and 3 are the change in BMI (kg/m²) over the 6-month intervention (v8-v2) plotted against the bile acid concentrations at baseline for each subject in the intervention group B420 (N=25) and Placebo (N=36). The median threshold is plotted as well.

From the confirmatory analysis (Table 4), using linear regression lines to illustrate the difference, it can be observed from FIGS. 2 and 3 that the placebo group is gaining weight/positive trendline for BMI, while the B420 group is losing weight/negative trendline for BMI during the 6 months intervention.

Secondary bile acids (e.g. LCA) are produced by bacteria in the microbiome and the secondary bile acids can be conjugated in the liver (e.g. GLCA).

Method description: Blood plasma Bile acid analysis—LC-MRM-MS

Fasting blood samples collected at both baseline and 6-month visits were used for analysis of plasma bile acids. Sample preparation was performed in triplicates, and samples were stored at −80° C. until analysis.

A mixture of 15 stable isotope labelled Bile acids was added to an Eppendorf tube and the solvent evaporated (Table 5).

TABLE 5
Deuterium labelled standards used for the LC-MRM-MS quantification of bile acids.

| Short name | CAS no | Bile acid | Vendor | Art no | CLP |
|---|---|---|---|---|---|
| D4-CA | 116380-66-6 | Cholic acid (2,2,4,4-D4, 98%) | Cambridge Isotope Lab | DLM-2611 | |
| D4-CDCA | 99102-69-9 | Chenodeoxychlic acid (2,2,4,4-D4, 98%) | Cambridge Isotope Lab | DLM-6780 | GHS08 |
| D4-DCA | 112076-61-6 | Deoxycholic acid (2,2,4,4-D4, 98%) | Cambridge Isotope Lab | DLM-2824 | GHS07 |
| D4-LCA | 83701-16-0 | Lithocholic acid (2,2,4,4-D4, 98%) | Cambridge Isotope Lab | DLM-9560 | |
| D4-HDCA | | — | — | | |
| D4-UDCA | 347841-46-7 | Ursodeoxycholic acid (2,2,4,4-D4, 98%) | Cambridge Isotope Lab | DLM-9574 | |
| D4-GCA | NA | Glycocholic acid (2,2,4,4-D4, 98%) | Cambridge Isotope Lab | DLM-2742 | GHS09 |
| D9-GCDCA | NA | Glycochenodeoxycholic acid (2,2,3,4,4,6,6,7,8-D9, 98%) | Cambridge Isotope Lab | DLM-9550 | |
| D4-GDCA | 1069132-37-1 | Glycodeoxycholic acid (2,2,4,4-D4, 98%) | Cambridge Isotope Lab | DLM-9554 | GHS07 |
| D4-GLCA | NA | Glycolithocholic acid (2,2,4,4-D4, 98%) | Cambridge Isotope Lab | DLM-9556 | GHS07 |
| D4-GHDCA | | — | — | | |
| D4-GUDCA | NA | Glycoursodeoxychloric acid (2,2,4,4-D4, 98%) | Cambridge Isotope Lab | DLM-9558 | |
| D4-TCA | 252030-90-3 | Taurocholic Acid-[D4] Na Salt | IsoSciences | 13232 | |
| D4-TCDCA | 6009-98-9 | Taurochenodeoxycholic acid Na Salt (2,2,4,4-D4, 98%) | Cambridge Isotope Lab | DLM-9562 | |
| D4-TDCA | 1180-95-6 | Taurodeoxycholic acid Na salt (2,2,4,4-D4, 98%) | Cambridge Isotope Lab | DLM-9568 | |
| D4-TLCA | 6042-32-6 | Taurolithocholic acid Na salt (2,2,4,4-D4, 98%) | Cambridge Isotope Lab | DLM-9570 | |
| D4-THDCA | | — | — | | |
| D4-TUDCA | 14605-22-2 | Tauroursodeoxycholic Acid-[D4] Na Salt | IsoSciences | 13106 | |

For each replicate 50 μL plasma were vortexed with 100 μL 1% aqueous formic acid and 350 μL acetonitrile was added. The tubes were sonicated for 10 min, and then kept on ice for 30 min to assist protein precipitation. After centrifugation at 16000×g for 2 min, the supernant was loaded on activated Phenomenex Phree Phospholipid Removal 96-well SPE plates The plate was centrifuged for 5 min at 500×g and the initial eluate was collected in a 1 mL 96-well collection plate. Each well was washed with 500 μL 70% aqueous acetonitrile containing 0.2% formic acid. The flow-through were pooled with the initial flow-through and dries in a vacuum concentrator. The sediment was reconstituted in 100 μL 50% aqueous methanol.

An Agilent 1290 UHPLC-system coupled to a TSQ Vantage from Thermo via a heated electrospray ionisation (HESI) source updated in negative mode. A Waters ACQUITY UPLC BEH C₁₈ (2.1 mm×150 mm, 1.7 μm, 100 Å) column was used with the gradient elution, with 0.01% formic acid in water (solvent A) and 0.01% formic acid in acetonitrile (solvent B) as mobile phase. The gradient was optimized from 25% to 40% B in 12 min and then 40% to 75% B in 14 min. The column was washed with 100% B 2 min and equilibrated with 25% B for 4 min between injections. The flow rate was 0.35 mL/min, and the column was maintained at 45° C. Injection volume was 10 μL.

The mass spectrometry settings for the TSQ instrument in the EZ-mode was: Negative mode, Spray voltage: 3000V, Capillary temperature: 320° C., Sheath gas: 45 psi, AUX gas 5, Vaporiser gas temperature: 300° C., Collision pressure: 1.3 mTorr, Cycle time: 0.800 s

Statistical analyses: See Example 1

### Example 4—Fecal Bacterial Genera Coprococcus and Ruminococcus Results

The relative abundance of bacterial genera Coprococcus and Ruminococcus identified by sequencing of 16S rRNA from feces were found to be significant predictors of weight gain reduction by the Latent Class Analysis model (Example 1). As both the predictive Z-score and Non-Responders Z-score are significant negative, and the Responders Z-score is negative but non-significant for both Coprococcus and Ruminococcus, it means that a high level of one or the other genera is predictive of reduced weight gain during a 6 months intervention with B420. The faecal relative abundance threshold for predictive weight gain reduction of subjects are in the ranges of [0.0360:0.0537] and [0.0319:0.0921] with median values of 0.0426 and 0.0631 for Coprococcus and Ruminococcus respectively. In FIGS. 4 and 5 are the change in BMI (kg/m²) over the 6-month intervention (v8-v2) plotted against the rel. abundance of Coprococcus and Ruminococcus for each subject in the intervention group B420 (N=25) and Placebo (N=36) as well as the thresholds.

From the confirmatory analysis (Table 4), using linear regression lines to illustrate the difference, it can be observed from FIGS. 4 and 5 that the placebo group is gaining weight/positive trendline for BMI, while the B420 group is losing weight/negative trendline for BMI during the 6 months intervention.

Method description: DNA Isolation and microbiota sequencing (From Hibberd 2019).

Microbial DNA was extracted from faecal samples with the MagMAX™ Total Nucleic Acid Isolation Kit (Applied Biosystems, Bridgewater, NJ, USA) and purified with the OneStep-96™ PCR Inhibitor Removal Kit (Zymo Research, Irvine, California, USA). Microbial DNA was amplified in triplicate PCR with primers 515F (5′-GTGCCAGCMGCCGCGGTAA) and 806R (5′-GGACTACHVGGGTWTCTAAT) targeting the V4 variable region of the 16S rRNA gene. The PCR amplification conditions were 95° C. for 3 min for initial DNA denaturation, followed by 30 cycles at 95° C. for 45 s, 55° C. for 60 s, and 72° C. for 90 s; and final extension of 10 min at 72° C. PCR products were purified, normalized and paired-end 2×250 bp reads were generated with the Illumina MiSeq system (Roy J. Carver Biotechnology Center, University of Illinois Urbana-Champaign).

Statistical analyses: See Example 1.

### Example 5—Faecal Bacterial Genera Akkermansia Results

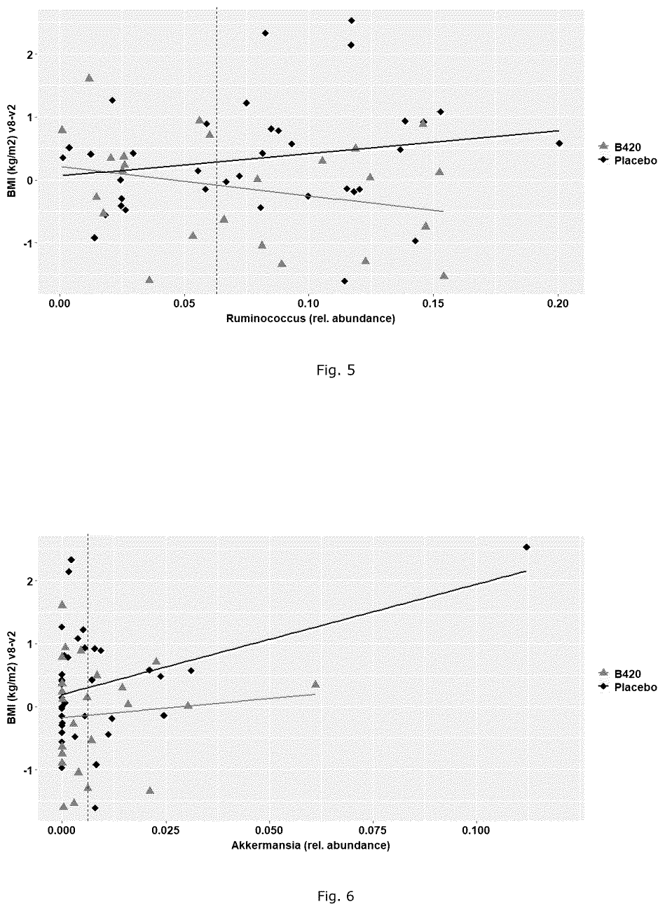

The relative abundance of bacterial genera Akkermansia identified by sequencing of 16S rRNA from faeces was found to be a significant predictor of weight gain reduction by the Latent Class Analysis model (Example 1). As both the predictive Z-score and Non-Responders Z-score are negative and significant, and the Responders Z-score is negative but non-significant for Akkermansia, it means that a high level of Akkermansia is predictive of reduced weight gain during a 6-month intervention with B420. The faecal relative abundance threshold for predictive weight gain reduction of subjects is in the range [0.0041:0.0118] with a median value of 0.0062 for Akkermansia. In FIG. 6 is depicted the change in BMI (kg/m²) over the 6-month intervention (v8-v2) plotted against the rel. abundance of Akkermansia for each subject in the intervention group B420 (N=25) and Placebo (N=36) as well as the threshold.

From the confirmatory analysis (Table 4), using linear regression lines to illustrate the difference, it can be observed from FIG. 6. that the placebo group is gaining weight/positive trendline for BMI, while the B420 group is also gaining weight/positive trendline for BMI, but to a lower extent than placebo during the 6 months intervention. The predictor genera Akkermansia predicts at baseline a lower weight gain/minor change of BMI for the 6-month intervention with B420 than the placebo group.

Method description: See Example 4.

### Example 6

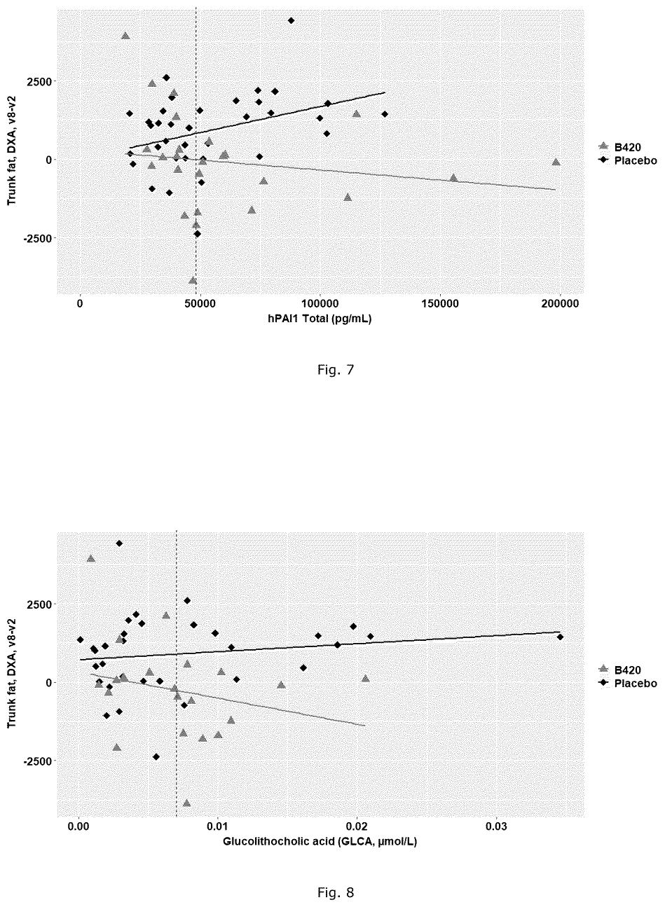

The total human plasminogen activator inhibitor-1 (hPAI1 Total) in blood samples was found to be a significant predictor of weight loss by the Latent Class Analysis model. As both the predictive Z-score and the responders Z-score are significant and negative, it means that a high level of hPAI1 Total is predictive of weight loss during a 6 months intervention with B420. The blood level threshold for predictive weight loss of subjects is in the range [40653:53294] pg/mL with a median of 48245 pg/mL (Table 4). In FIG. 7 is the change in Trunk fat by DXA scans over the 6-month intervention (v8-v2) plotted against hPAI1 Total at baseline for each subject in the intervention group B420 (N=25) and Placebo (N=36). The median hPAI1 Total threshold are plotted as well.

Using linear regression lines to illustrate the difference, it can be observed from FIG. 7 that the placebo group is gaining weight/positive increase in Trunk fat, while the B420 group is losing Trunk fat/negative trendline for Trunk fat during the 6 months intervention.

Method description: Thrunk fat, Android fat and Total fat is measured by Dual Energy X-ray absorptiometry (DXA) scans as described in Stenman 2016—change measured in grams.

HPAI1 Total determination & Statistical analysis: Example 2.

### Example 7

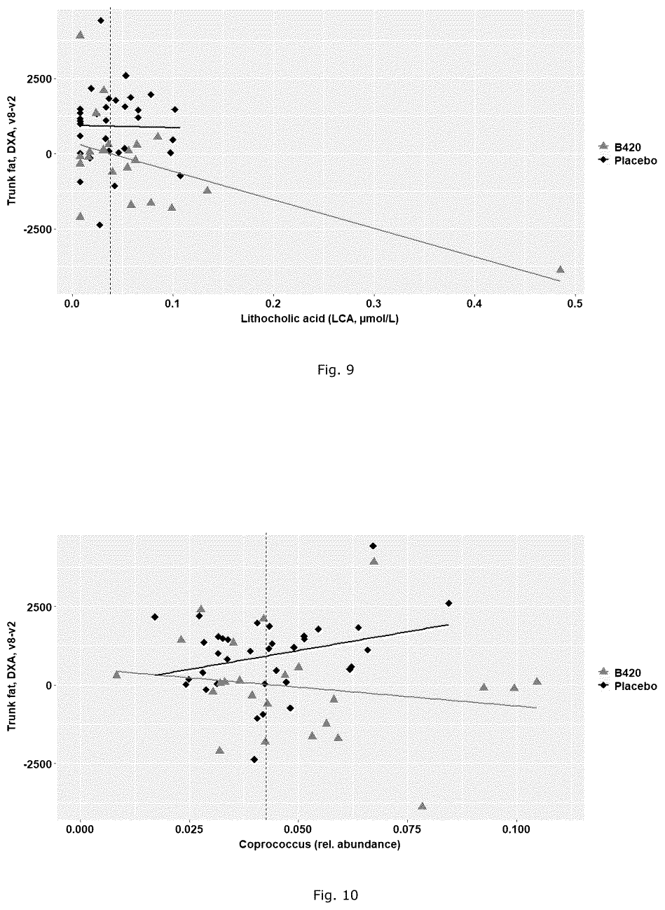

The secondary bile acids Glucolithocholic acid (GLCA) and Lithocholic acid (LCA) in blood samples were found to be significant predictors of weight loss by the Latent Class Analysis model. As both the predictive Z-score and Responders Z-score are significant and negative for both GLCA and LCA, it means that a high level of one or the other secondary bile acid is predictive of weight loss during the 6-month intervention with B420. The blood level threshold for predictive weight loss of subjects should be in the ranges [0.0033:0.0078] and [0.0301:0.0583] with median values of 0.0070 μmol/L and 0.0380 μmol/L for GLC and LCA respectively. In FIGS. 8 and 9 are the change Trunk fat by DXA scans over the 6-month intervention (v8-v2) plotted against the bile acid concentrations at baseline for each subject in the intervention group B420 (N=25) and Placebo (N=36). The median thresholds for GLCA and LCA are plotted as well.

From the confirmatory analysis (Table 4), using linear regression lines to illustrate the difference, it can be observed from FIGS. 8 and 9 that the placebo group is gaining Trunk fat/positive trendline for Trunk fat, while the B420 group is losing Trunk fat/negative trendline during the 6 months intervention.

Method description: Bile acid determination: See Example 3. DXA scan method: See Example 6. Statistical analyses: See Example 1.

### Example 8

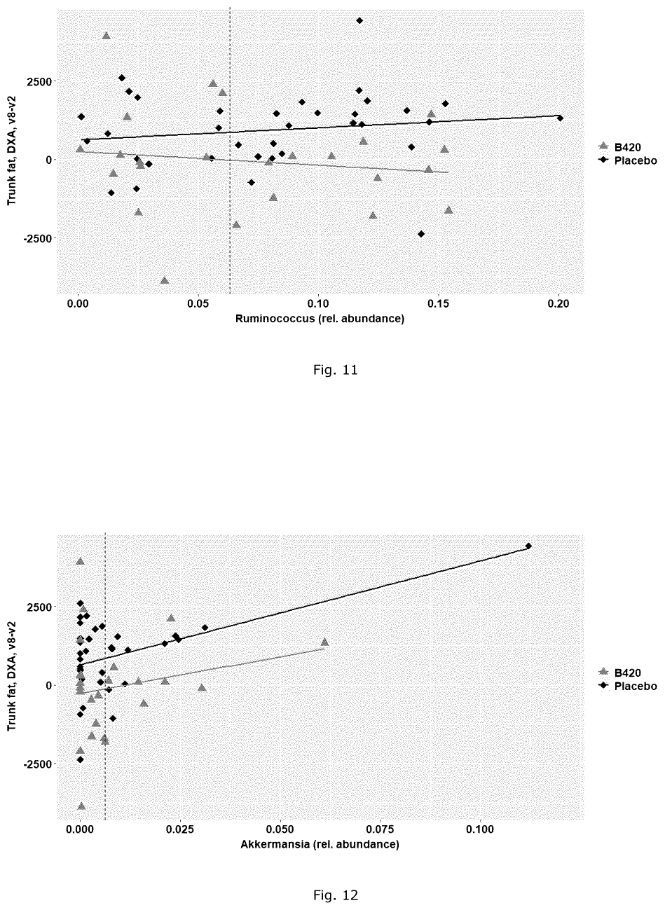

The relative abundance of bacterial genera Coprococcus and Ruminococcus identified by sequencing of 16S rRNA from faeces were found to be significant predictors of weight gain reduction by the Latent Class Analysis model (Example 1). As both the predictive Z-score and Non-Responders Z-score are significant negative, and the Responders Z-score is negative but non-significant for both Coprococcus and Ruminococcus, it means that a high level of one or the other genera is predictive of reduced weight gain during a 6 months intervention with strain B420. The faecal relative abundance threshold for predictive weight gain reduction of subjects are in the ranges of [0.0360:0.0537] and [0.0319:0.0921] with median values of 0.0426 and 0.0631 for Coprococcus and Ruminococcus respectively. In FIGS. 10 and 11 are the change in Trunk fat measured by DXA scans over the 6-month intervention (v8-v2) plotted against the relative abundance of Coprococcus and Ruminococcus for each subject in the intervention group B420 (N=25) and Placebo (N=36) as well as the thresholds.

From the confirmatory analysis (Table 4), using linear regression lines to illustrate the difference, it can be observed from FIGS. 10 and 11 that the placebo group is gaining Trunk fat/positive trendline for BMI, while the B420 group is losing Trunk fat/negative trendline during the 6 months intervention.

Method description: Faecal microbiome 16S rRNA sequencing: See Example 4. DXA scan method: See Example 6. Statistical analyses: See Example 1.

### Example 9

The relative abundance of bacterial genera Akkermansia identified by sequencing of 16S rRNA from faeces was found to be a significant predictor of weight gain reduction by the Latent Class Analysis model (Example 1). As both the predictive Z-score and Non-Responders Z-score are negative and significant, and the Responders Z-score is negative but non-significant for Akkermansia, it means that a high level of Akkermansia is predictive of reduced weight gain during a 6-month intervention with B420. The fecal relative abundance threshold for predictive weight gain reduction of subjects is in the range [0.0041:0.0118] with median=0.0062 for Akkermansia. In FIG. 12 is the change in Trunk fat over the 6-month intervention (v8-v2) plotted against the rel. abundance of Akkermansia for each subject in the intervention group B420 (N=25) and Placebo (N=36) as well as the median threshold.

From the confirmatory analysis (Table 4), using linear regression lines to illustrate the difference, it can be observed from FIG. 12. that the placebo group is gaining Trunk fat/positive trendline, while the B420 group is also gaining Trunk fat/positive trendline, but to a lower extent than placebo during the 6 months intervention. The predictor genera Akkermansia predicts at baseline a lower Trunk fat gain for the 6-month intervention with B420 than the placebo group.

Method description: See Example 8.

### Example 10

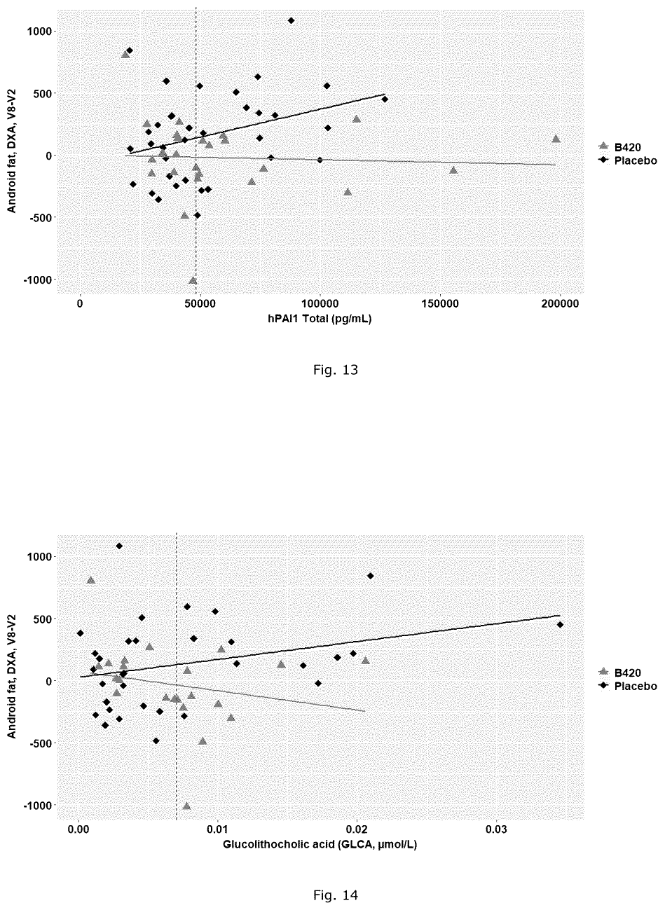

The total human plasminogen activator inhibitor-1 (hPAI1 Total) in blood samples was found to be a significant predictor of weight loss by the Latent Class Analysis model. As both the predictive Z-score and the responders Z-score are significant and negative, it means that a high level of hPAI1 Total is predictive of weight loss during a 6 months intervention with B420. The blood level threshold for predictive weight loss of subjects is in the range [40653:53294] pg/mL with a median of 48245 pg/mL (Table 4). In FIG. 13 is the change in Android fat by DXA scans over the 6-month intervention (v8-v2) plotted against hPAI1 Total at baseline for each subject in the intervention group B420 (N=25) and Placebo (N=36). The median hPAI1 Total threshold is plotted as well.

From the confirmatory analysis (Table 4), using linear regression lines to illustrate the difference, it can be observed from FIG. 13 that the placebo group is gaining Android fat/positive trendline, while the B420 group is losing Android fat/negative trendline during the 6 months intervention.

Method description: See Example 6.

### Example 11

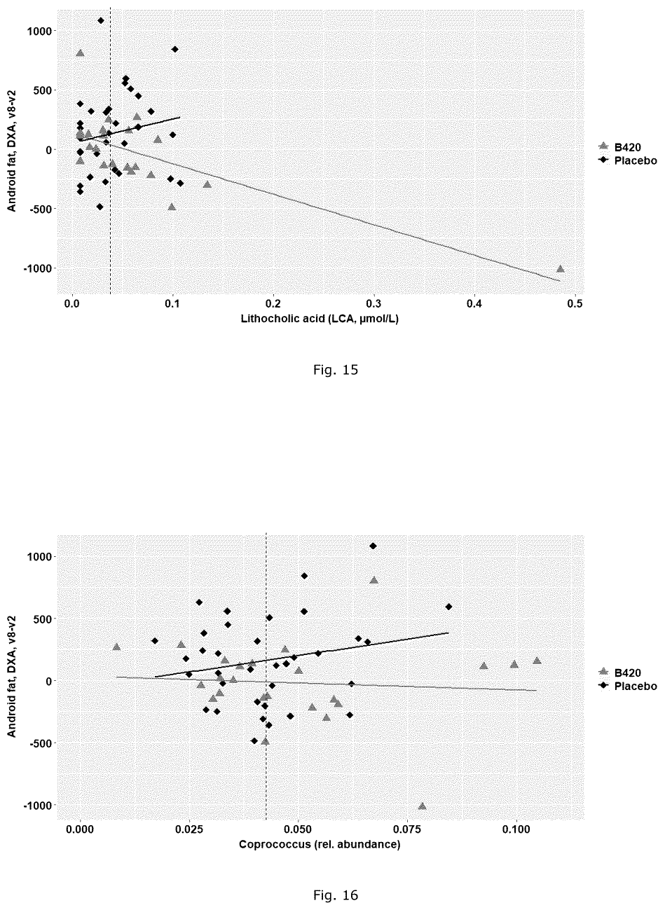

The secondary bile acids Glucolithocholic acid (GLCA) and Lithocholic acid (LCA) in blood samples were found to be significant predictors of weight loss by the Latent Class Analysis model. As both the predictive Z-score and Responders Z-score are significant and negative for both GLCA and LCA, it means that a high level of one or the other secondary bile acid is predictive of weight loss during the 6-month intervention with B420. The blood level threshold for predictive weight loss of subjects should be in the ranges [0.0033:0.0078] and [0.0301:0.0583] with median values of 0.0070 μmol/L and 0.0380 μmol/L for GLC and LCA respectively. In FIGS. 14 and 15 are the change in Android fat by DXA scans over the 6-month intervention (v8-v2) plotted against the bile acid concentrations at baseline for each subject in the intervention group B420 (N=25) and Placebo (N=36). The median thresholds for GLCA and LCA are plotted as well.

From the confirmatory analysis (Table 4), using linear regression lines to illustrate the difference, it can be observed from FIGS. 14 and 15 that the placebo group is gaining Android fat/positive trendline, while the B420 group is losing Android fat/negative trendline during the 6 months intervention.

Method description: See Example 7.

### Example 12

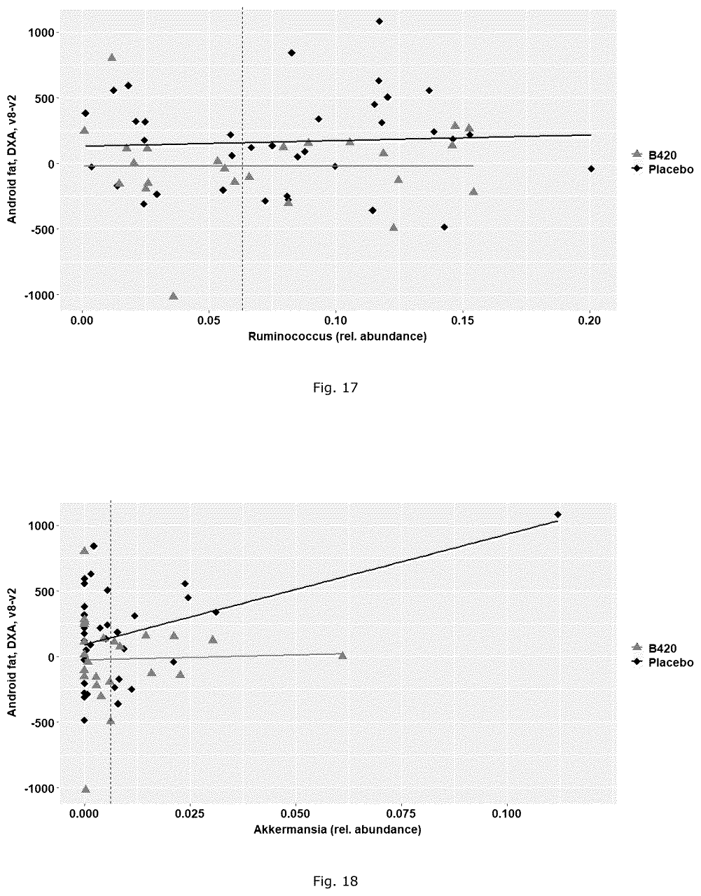

The relative abundance of bacterial genera Coprococcus and Ruminococcus identified by sequencing of 16S rRNA from feces were found to be significant predictors of weight gain reduction by the Latent Class Analysis model (Example 1). As both the predictive Z-score and Non-Responders Z-score are significant negative, and the Responders Z-score is negative but non-significant for both Coprococcus and Ruminococcus, it means that a high level of one or the other genera is predictive of reduced weight gain during a 6 months intervention with B420. The fecal relative abundance threshold for predictive weight gain reduction of subjects are in the ranges of [0.0360:0.0537] and [0.0319:0.0921] with median values of 0.0426 and 0.0631 for Coprococcus and Ruminococcus respectively. In FIGS. 16 and 17 are the change in Android fat over the 6-month intervention (v8-v2) plotted against the rel. abundance of Coprococcus and Ruminococcus for each subject in the intervention group B420 (N=25) and Placebo (N=36) as well as the median thresholds.

From the confirmatory analysis (Table 4), using linear regression lines to illustrate the difference, it can be observed from FIGS. 16 and 17 that the placebo group is gaining Android fat/positive trendline, while the B420 group is losing Android fat/negative trendline during the 6 months intervention. The predictive effect on loss of Android fat of genera Ruminococcus is less pronounced than that of Coprococcus.

Method description: See Example 8

### Example 13

The relative abundance of bacterial genera Akkermansia identified by sequencing of 16S rRNA from faeces was found to be a significant predictor of weight gain reduction by the Latent Class Analysis model (Example 1). As both the predictive Z-score and Non-Responders Z-score are negative and significant, and the Responders Z-score is negative but non-significant for Akkermansia, it means that a high level of Akkermansia is predictive of reduced weight gain during a 6-month intervention with B420. The faecal relative abundance threshold for predictive reduced weight gain of subjects is in the range [0.0041:0.0118] with median=0.0062 for Akkermansia. In FIG. 18 is the change in Android fat over the 6-month intervention (v8-v2) plotted against the relative abundance of Akkermansia for each subject in the intervention group B420 (N=25) and Placebo (N=36) as well as the median threshold.

From the confirmatory analysis (Table 4), using linear regression lines to illustrate the difference, it can be observed from FIG. 18 that the placebo group is gaining Trunk fat/positive trendline, while the B420 group is also gaining Trunk fat/positive trendline, but to a lower extent than placebo during the 6 months intervention. The predictor genera Akkermansia predicts at baseline a lower change of Android fat gain for the 6-month intervention with B420 than the placebo group.

Method description: See Example 8

### Example 14

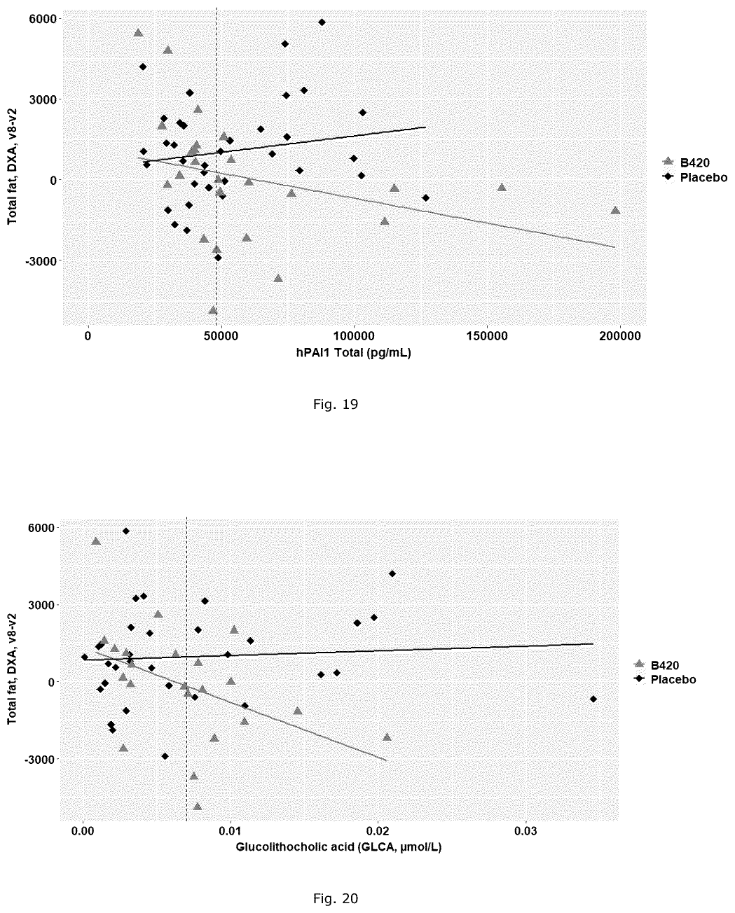

The total human plasminogen activator inhibitor-1 (hPAI1 Total) in blood samples was found to be a significant predictor of weight loss by the Latent Class Analysis model. As both the predictive Z-score and the responders Z-score are significant and negative, it means that a high level of hPAI1 Total is predictive of weight loss during a 6 months intervention with B420. The blood level threshold for predictive weight loss of subjects is in the range [40653:53294] pg/mL (33; 66 percentiles) with a median of 48245 pg/mL. In FIG. 19 is the change in Total fat by DXA scans over the 6-month intervention (v8-v2) plotted against hPAI1 Total at baseline for each subject in the intervention group B420 (N=25) and Placebo (N=36). The median hPAI1 Total threshold is plotted as well.

From the confirmatory analysis (Table 4), using linear regression lines to illustrate the difference, it can be observed from FIG. 19 that the placebo group is gaining Total fat/positive trendline, while the B420 group is losing Total fat/negative trendline during the 6 months intervention.

Method description: See Example 6.

### Example 15

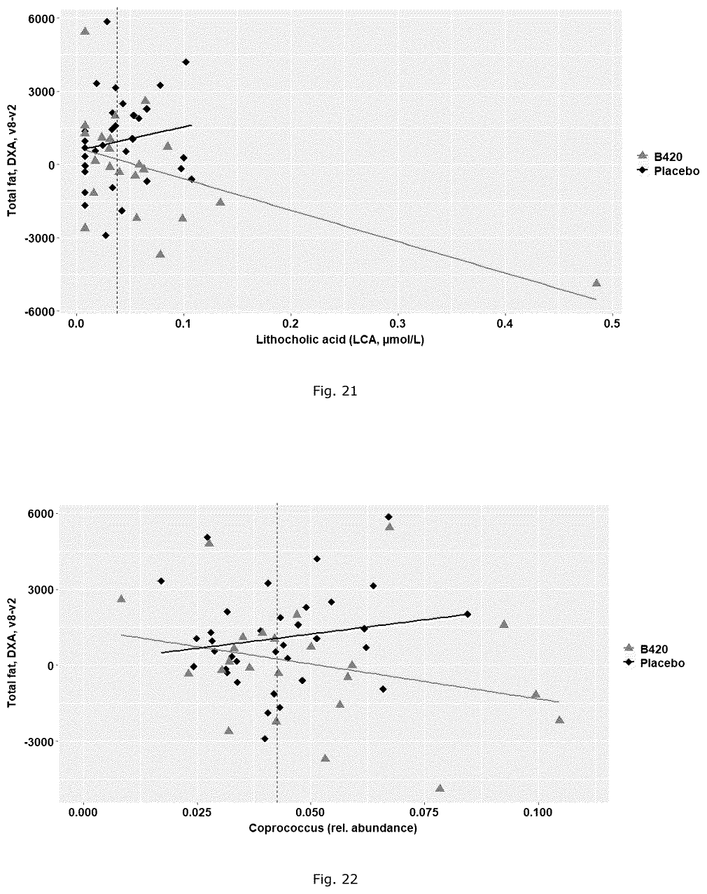

The secondary bile acids Glucolithocholic acid (GLCA) and Lithocholic acid (LCA) in blood samples were found to be significant predictors of weight loss by the Latent Class Analysis model. As both the predictive Z-score and Responders Z-score are significant and negative for both GLCA and LCA, it means that a high level of one or the other secondary bile acid is predictive of weight loss during the 6-month intervention with B420. The blood level threshold for predictive weight loss of subjects should be in the ranges [0.0033:0.0078] and [0.0301:0.0583] with median values of 0.0070 μmol/L and 0.0380 μmol/L for GLC and LCA respectively. In FIGS. 20 and 21 are the change in Total fat by DXA scans over the 6-month intervention (v8-v2) plotted against the bile acid concentrations at baseline for each subject in the intervention group B420 (N=25) and Placebo (N=36). The median thresholds for GLCA and LCA are plotted as well.

From the confirmatory analysis (Table 4), using linear regression lines to illustrate the difference, it can be observed from FIGS. 20 & 21 that the placebo group is gaining Total fat/positive trendline, while the B420 group is losing Total fat/negative trendline during the 6 months intervention.

Method description: See Example 7.

### Example 16

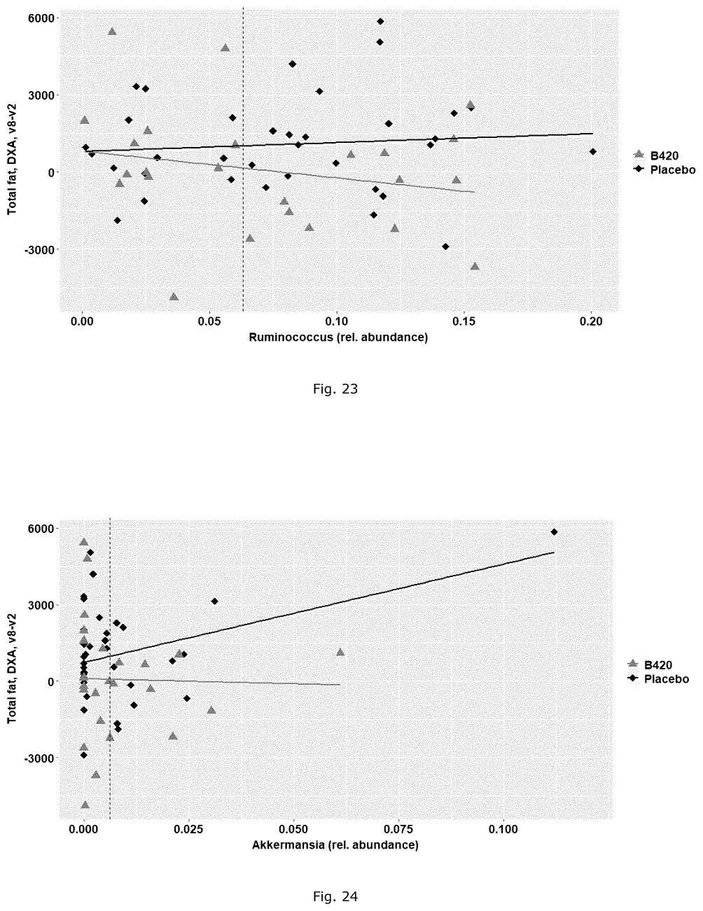

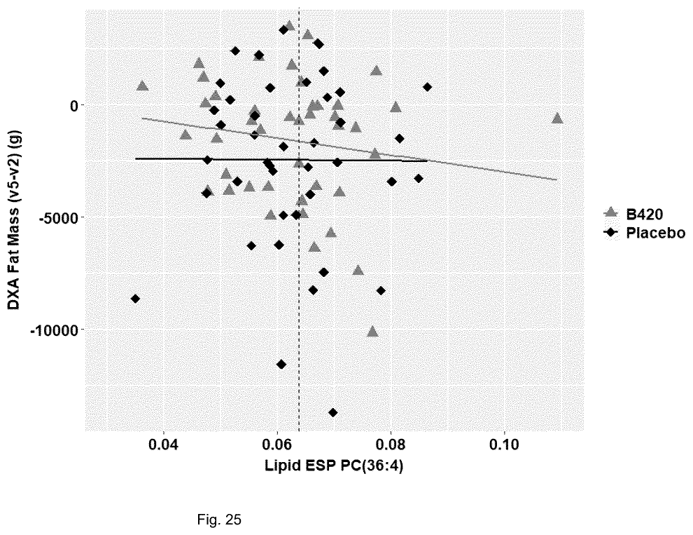

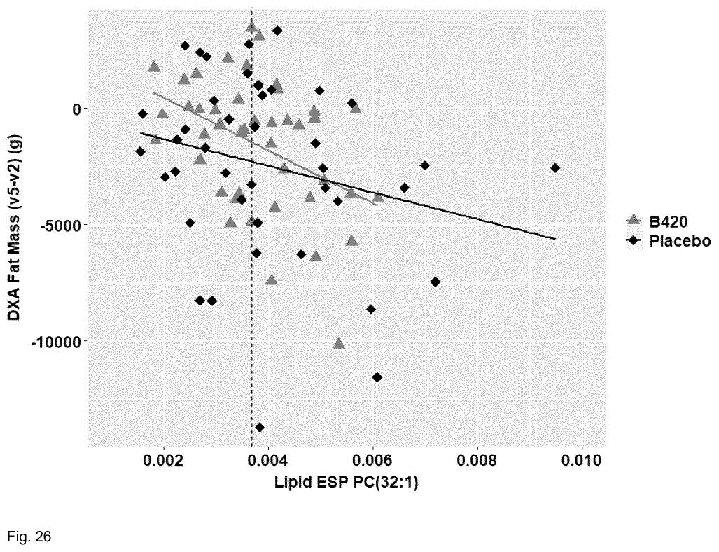

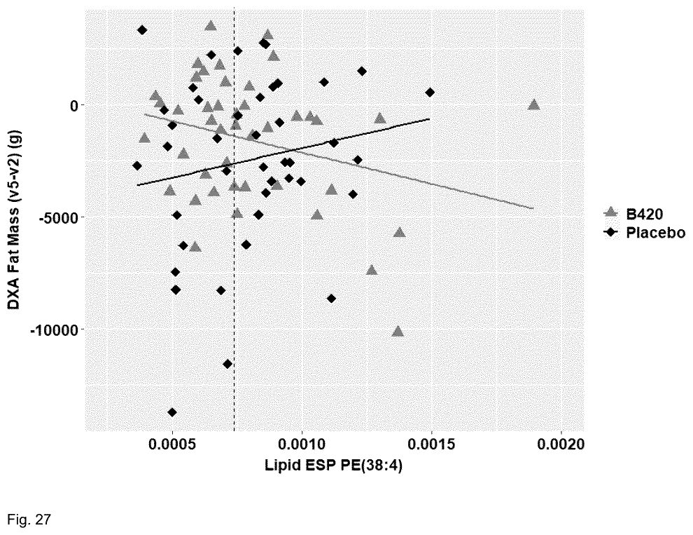

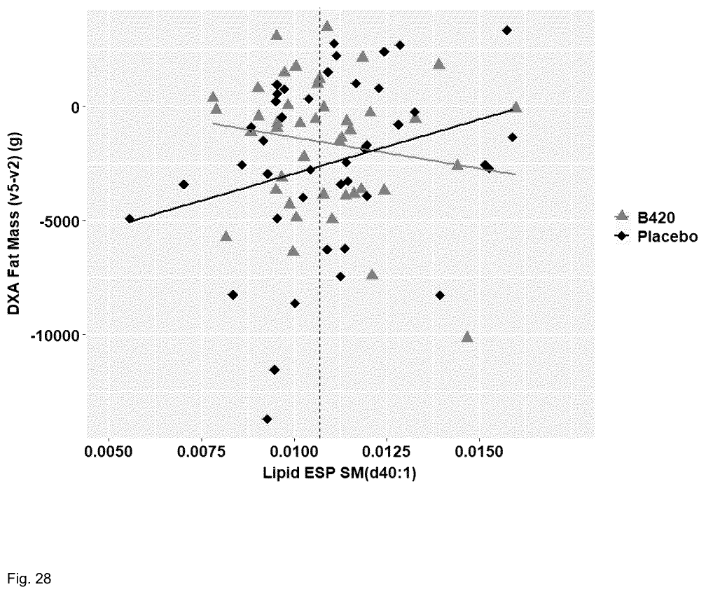

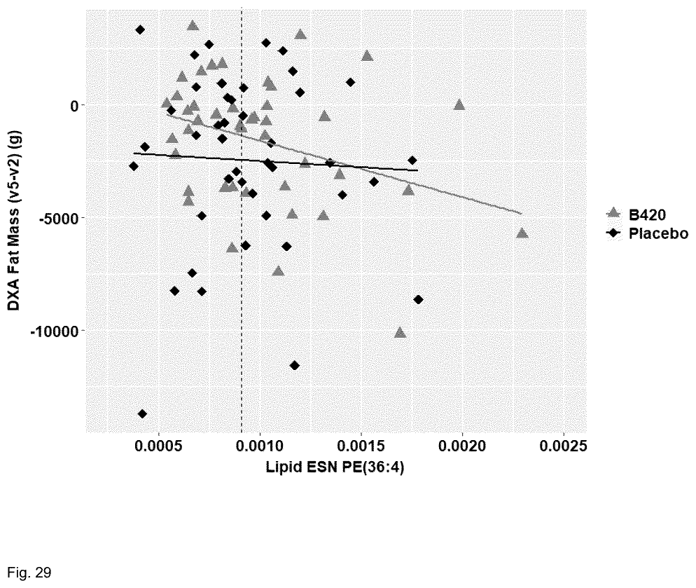

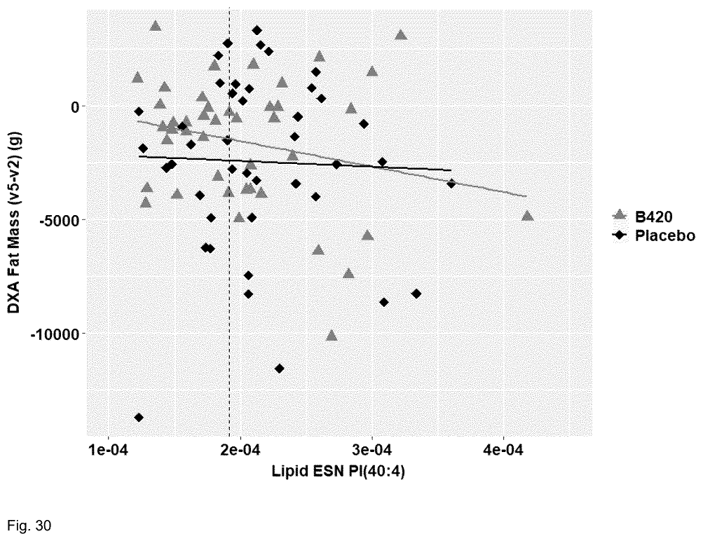

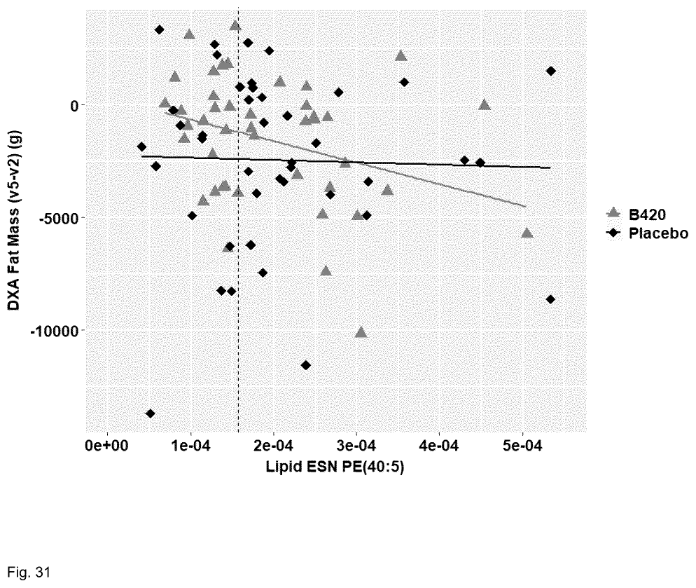

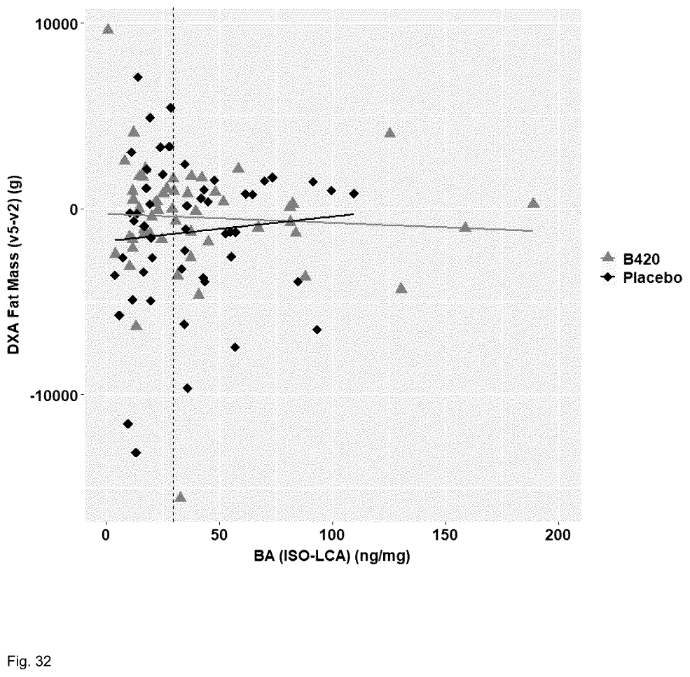

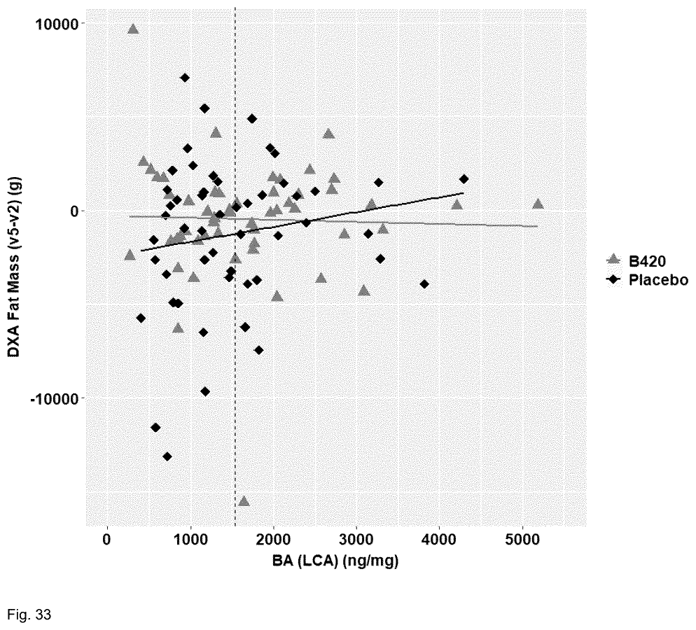

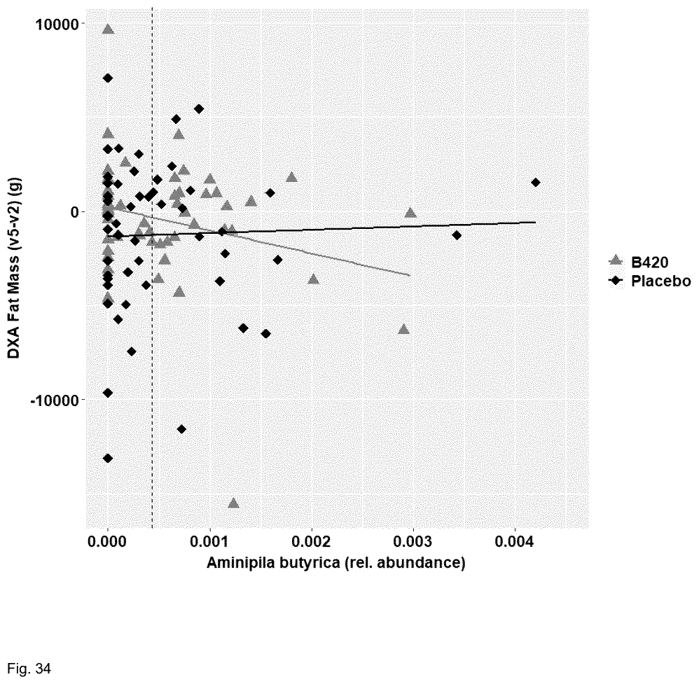

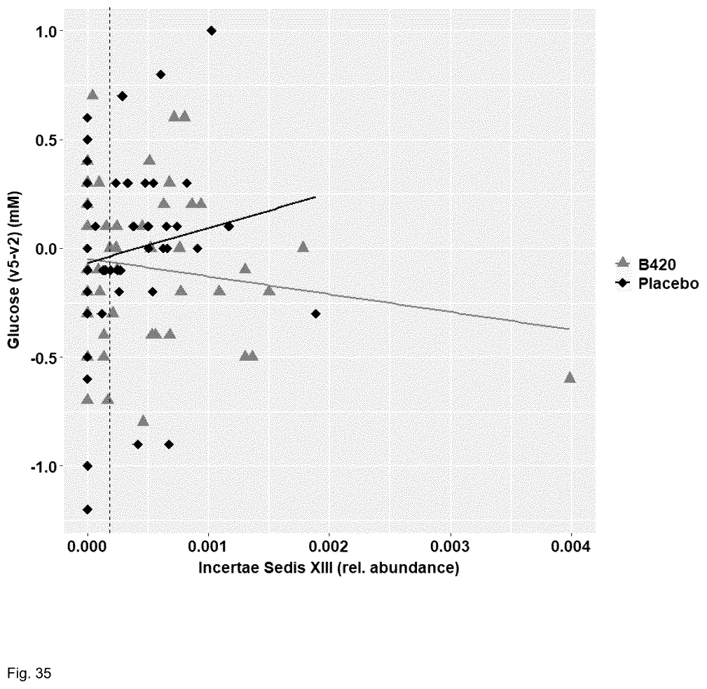

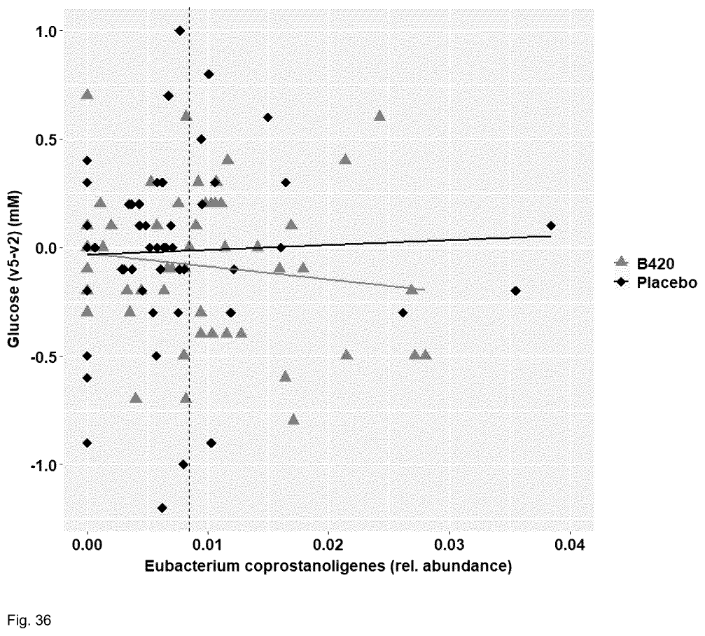

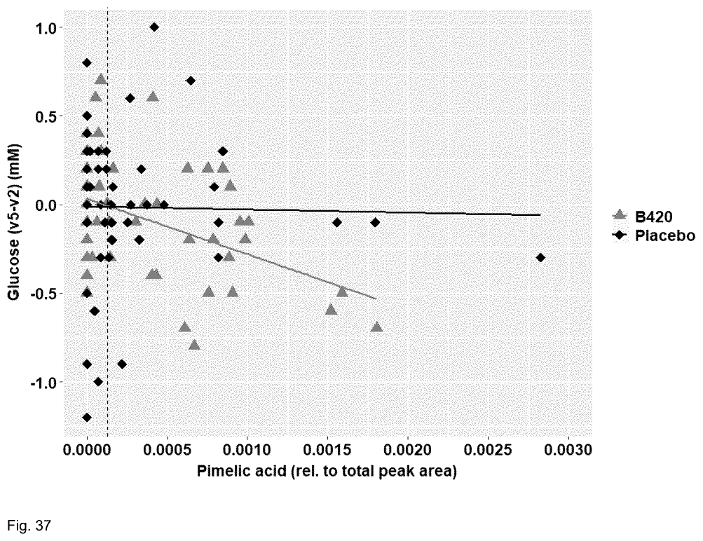

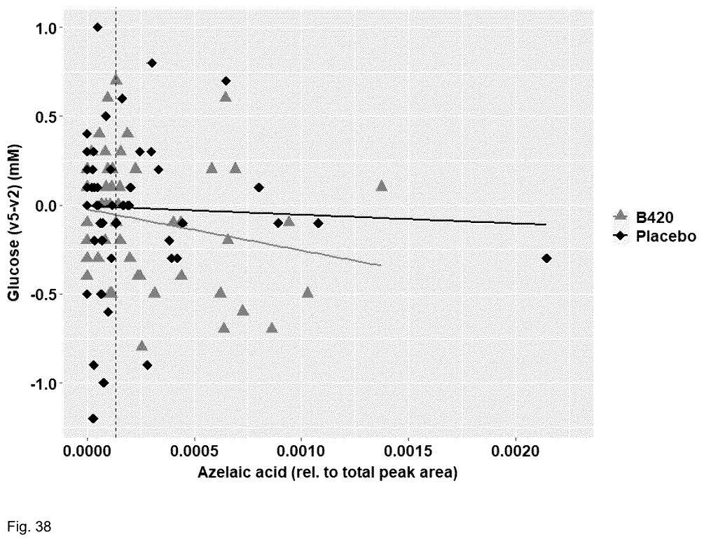

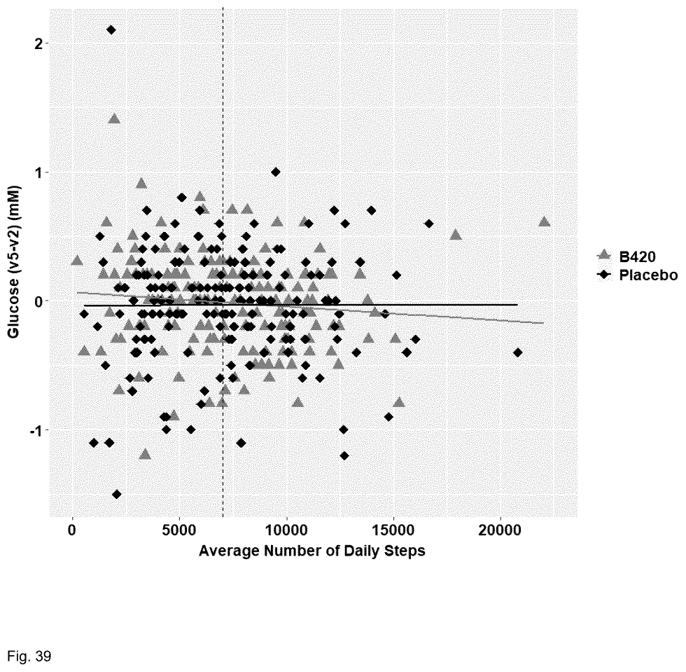

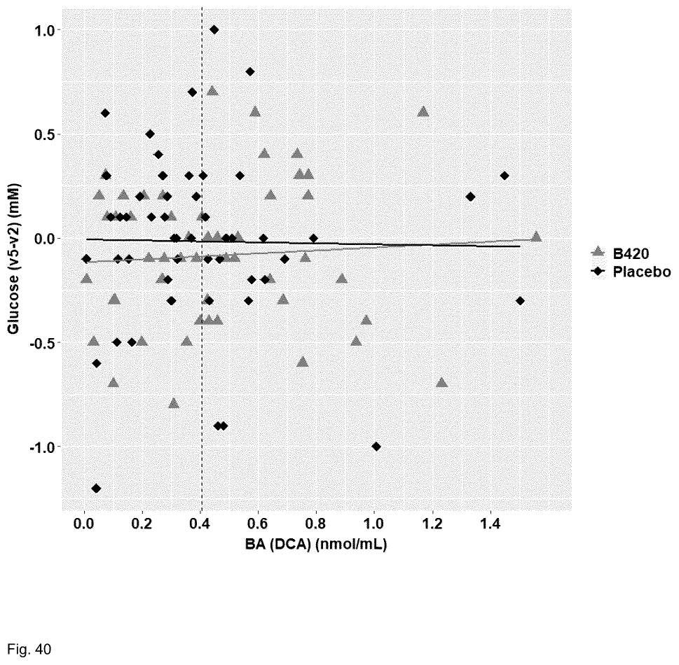

The relative abundance of bacterial genera Coprococcus and Ruminococcus identified by sequencing of 16S rRNA from feces were found to be significant predictors of weight gain reduction by the Latent Class Analysis model (Example 1). As both the predictive Z-score and Non-Responders Z-score are significant negative, and the Responders Z-score is negative but non-significant for both Coprococcus and Ruminococcus, it means that a high level of one or the other genera is predictive of reduced weight gain during a 6 months intervention with B420. The fecal relative abundance threshold for predictive weight gain reduction of subjects are in the ranges of [0.0360:0.0537] and [0.0319:0.0921] with median values of 0.0426 and 0.0631 for Coprococcus and Ruminococcus respectively. In FIGS. 22 and 23 are the change in Total fat over the 6-month intervention (v8-v2) plotted against the rel. abundance of Coprococcus and Ruminococcus for each subject in the intervention group B420 (N=25) and Placebo (N=36) as well as the median thresholds.

From the confirmatory analysis (Table 4), using linear regression lines to illustrate the difference, it can be observed from FIGS. 22 and 23 that the placebo group is gaining Total fat/positive trendline, while the B420 group is losing Total fat/negative trendline during the 6 months intervention.

Method description: See Example 8.

### Example 17

The relative abundance of bacterial genera Akkermansia identified by sequencing of 16S rRNA from feces was found to be a significant predictor of weight gain reduction by the Latent Class Analysis model (Example 1). As both the predictive Z-score and Non-Responders Z-score are negative and significant, and the Responders Z-score is negative but non-significant for Akkermansia, it means that a high level of Akkermansia is predictive of reduced weight gain during a 6-month intervention with B420. The fecal relative abundance threshold for predictive Total fat change of subjects is in the range [0.0041:0.0118] with median=0.0062 for Akkermansia. In FIG. 24 is the change in Total fat over the 6-month intervention (v8-v2) plotted against the rel. abundance of Akkermansia for each subject in the intervention group B420 (N=25) and Placebo (N=36) as well as the median threshold.

From the confirmatory analysis (Table 4), using linear regression lines to illustrate the difference, it can be observed from FIG. 24 that the placebo group is gaining Total fat having a positive trendline, while the B420 group is losing Total fat having a negative trendline during the 6 months intervention.

Method description: See Example 8.

### Example 18

The SlimCap_HL post hoc study aims at identifying baseline biomarkers that are associated to responder status (during intervention period) of individual subjects receiving an active treatment (B420) compared to a Placebo treatment.

### Clinical Study Design

The dataset for responder analysis is from SlimCap HL clinical trial. SlimCap HL was a randomized, triple-blind, placebo-controlled, multi-center, parallel group study on individuals with overweight or obesity following healthy lifestyle consisting of a recommended calorie-reduced diet (20% calorie restriction) and increase in daily activity (1000 steps more per day). The clinical study consisted of five visits: screening visit (visit 1), baseline visit (visit 2), and 2-month, 4-month and 6-month post baseline visits (visits 3, 4 and 5, respectively), of which only baseline (visit 2, v2) and 6 months data was used in the responder analysis. At baseline, healthy individuals with overweight or obesity from France and Spain were divided into two groups, Placebo and B420™. B420™ group were supplemented with Bifidobacterium animalis ssp. lactis 420 at a target dose of 1×10¹⁰ colony forming units (CFU) in a capsule with micro-crystalline cellulose (MCC), 1% magnesium stearate and 1% silicon dioxide taken orally once per day for 6 months (Batch No: 1103400800 [2.07×1010 CFU/capsule], 1103714113 [1.92×1010 CFU/capsule] and 1103840294 [1.47×1010 CFU/capsule]). Placebo group was supplemented with capsules containing microcrystalline cellulose (MCC), 1% magnesium stearate and 1% silicon dioxide) taken orally once per day for 6 months, Batch No: 1103400790, 1103714112 and 1103840293). There were 209 participants in each group, 418 in total in the study.

The purpose of this study is to demonstrate whether B420™ can reduce total body fat mass more than placebo when participants follow a healthy lifestyle intervention. The study is a confirmatory for previous clinical study MetsProb in a larger population with only probiotic and a healthy lifestyle program consisting of calorie restriction and increase in daily activity.

The population for this study included participants with overweight and obesity, especially abdominal obesity, who were not pharmacologically treated for metabolic syndrome or related diseases and were not taking drugs or supplements to manage body weight or body fat. Participants were asked to follow healthy lifestyle habits for the duration of the study.

Primary endpoint of the study was difference in relative change in total body fat mass from baseline (Visit 2) to 6 months of product intake (Visit 5) between the active and placebo group.

Secondary variables were differences between the active vs. placebo group for:
Fat mass in the trunk region of the body (DXA)
Waist circumference
Fat mass in the android region of the body (DXA)
Lean body mass (DXA)
Energy intake
Fat mass in other regions of the body (arms, legs and gynoid) (DXA)
Absolute total body fat mass (DXA)
Body weight
Body mass index (BMI)
Hip circumference
Relative total body fat mass at 2 and 4 months (DXA)
Percentage total body fat mass (added during SAP development)

Ancillary variables were differences between the active vs. placebo group for:
Food intake
Physical activity (IPAQ, see Appendix 2)
Daily activity (daily steps, pedometer/accelerometer)

Exploratory variables were differences between the active vs. placebo group for:
Fasting glucose
Fasting insulin
Insulin resistance
Glycated hemoglobin HbA1c
Blood lipids (total cholesterol, HDL, LDL, triglycerides)
Inflammation markers
Circulating zonulin
Markers of gut barrier function and endotoxemia
Fecal microbiota
Fecal metabolites
Adipose tissue biomarkers

At visit 2 (baseline, v2), after checking the 3-day Food Diary and pedometer/accelerometer record, dietary counselling to instruct the participants to follow a healthy lifestyle intervention was given. Calorie restriction of 20% of Total Energy Expenditure (TEE) was instructed, as well as an increase in daily activity (1000 steps more per day compared to baseline). The 20% calorie restriction was calculated from TEE based on BMR and Physical Activity Level (PAL) based on IPAQ at Visit 1 according to FAO.

Body fat and lean mass were measured at Visits 2, 3, 4 and 5 using dual-energy X-ray absorptiometry (DXA). DXA measurement occurred the same day or as close as possible to the site visit and maximum 3 days after the visit. The waist circumference was measured at the midpoint between the lower margin of the last palpable rib and the top of the iliac crest. Hip circumference was measured around the widest portion of the buttocks, with the tape parallel to the floor.

Blood samples were collected at Visits 2 and 5 for analysis of biomarkers. Blood glucose, HbA1c, and lipid parameters were also measured at screening (Visit 1) for inclusion/exclusion criteria purposes. In addition, circulating zonulin, a marker related to intestinal permeability, was analyzed from all the participants. To explore the possible mechanisms of action, other biomarkers related to gut barrier function and endotoxemia (LPS, sCD14) and adipose tissue metabolism (adiponectin), and systemic inflammation biomarkers (Glycosylated blood proteins, GlycA/GlycB) by NMR were analyzed from all the participants as exploratory parameters. Advanced analytics methods were utilized to analyze a broad range of metabolite levels in the blood that serve as inflammation and adipose tissue metabolism markers.

Fecal samples were to be collected from at least 50 participants per study group, and overall, 220 fecal samples were collected, 115 at V2 and 105 at V5. Fecal microbiota composition and activity were analyzed using state-of the-art methods including 16S sequencing, quantitative PCR, and determination of bacterial metabolites (e.g. SCFAs, BCFAs). In addition, fecal zonulin, a host-related marker of gut health was measured as exploratory parameter.

### Analytical Methods—Additional Methods to the One's Used in MetSProb.

### LC-MS Lipids in Blood Serum.

Method is inspired by literature references Satomi Y. et al. 2017, Cajka T and Fiehn O 2014, and Sanjoy K. Bhattacharva 2017.

### 1.1. Chemicals and Reagents

Acetonitorile (LC/MS grade), ethanol (99.5%, HPLC grade), 2-propanol (LC/MS grade), formic acid (LC/MS grade), ammoniumacetate were purchased from Thermo Fisher Scientific Inc. (Kamstrup, Denmark).

### 1.2. Sample Preparation

Human serum (20 μL) was aliquoted out in triplicates in 1.5 mL tubes and 180 μL 90% ethanol (5° C.) was added followed by mixing at 5° C. for 5 min. After centrifugation at 14,000 rpm at 20° C. for 5 min, 160 μL of the supernatant was used for liquid chromatography/mass spectrometry (LC/MS) analysis.

### 1.3. Liquid Chromatography/Mass Spectrometry

The liquid chromatography/mass spectrometry system consisted of a Vanquish UHPLC (Thermo Fisher Scientific inc., Sunnyvale, CA, USA) and an Orbitrap Fusion MS system (Thermo Fisher Scientific inc., Sunnyvale, CA, USA) Liquid chromatography separation was performed at 65° C. using a reverse phase column, Acquity UPLC CSH C18 (1.7 μm, 100×2.1 mm and an Acquity UPLC CSH C18 1.7 μm VanGuard™, Waters co., Milford, MA, USA). The mobile phases consisted of 600:400 (v/v) acetonitrile/water with 10 mM ammonium acetate (mobile phase A), and a 900:100 (v/v) isopropanol/acetonitrile with 10 mM ammonium acetate (mobile phase B). A gradient elution was used for the lipid separation by the following program. The flow rate was set to 0.5 mL/min and the ratio of the mobile phase B was started at 15% B immediately increased to 30% B at 2.4 min, increased to 48% B at 3.0 min, increased to 82% B at 13.2 min, increased to 99% B at 13.8 min, maintained at 99% B for 0.6 min, then decreased to 15% B at 14.5 and kept constant for 3.5 min. Mass spectrometry analysis was performed separately in both positive (ESP) and negative ionization modes (ESN). The eluent from liquid chromatography was introduced directly for electrospray ionization using a heated electrospray ionization probe (H-ESI, Thermo Fisher Scientific

inc., San Jose, CA, USA) with a spray voltage at 2.5 kV for negative or 3.5 kV for positive ionization mode, and a vaporizer temperature at 350° C. Ion Transfer Tube was set at 325° C. Full mass spectra (MS) were acquired by an Orbitrap ranging from m/z 170 to m/z 1700 with a mass resolving power of 120000 FWHM (Full width at half maximum) at m/z 400. The product ion spectra (MS/MS) were obtained by Higher-energy C-trap dissociation (HCD).
1.4. Peak Extraction from LC/MS Raw Data

The raw data from the LC/MS were processed by LipidSearch™ 5.0 Thermo Fisher Scientific. First a product search of the raw data files was performed in LipidSearch by the following procedure:
1. Processes the Extracted Ion Chromatogram [EIC] and detects peaks [Separated Peak Area: SPA] for each lipid ion.
2. Collects the MS2 spectra contained in the EIC region.
3. Calculates the Product Ion Chromatogram [PIC] of the product ion peak in each MS2 spectrum and calculates the virtual spectrum [Deconvoluted MS2 Spectrum: DMS] distributed from the shape belonging to the SPA.
4. Lists lipid ions with matching DMS and peak patterns as candidates.

Following the product search an alignment processing procedure is done by LipidSearch.

Alignment is carried out in the following three steps.
Retention time correction
Lipid molecule peak grouping
Validation

The peak areas of the annotated compounds were exported as a CSV-file along with the monoisotopic m/z value and retention time information, and further data processing was performed using Excel and different statistical tools. The relative amounts used for threshold and figures are calculated as peak area for a given phospholipid divided with the total peak area of all identified lipids in either negative (ESN) or positive mode (ESP).

### GC-MS Analysis of Metabolites in Feces

The derivatization and GC-MS analytical method is based on literature reference 4.

### Materials and Chemicals

Absolute ethanol was purchased from VWR. Pyridine (>99.5%) was from Fischer Scientific and chloroform (>99.9%), ethyl chloroformate (>98%), sodium hydroxide, sodium bicarbonate, and sodium sulphate were obtained from Sigma Aldrich.

### GC-MS

The fecal aqueous extracts were subjected to derivatization with ethyl chloroformate (ECF) prior to analysis with GC-TOFMS. The ECF reacts with carboxylic acids, amines, and phenols to produce esters, carbamates, and carbonates, respectively. Samples were derivatized and analyzed in duplicates except for 13 samples that were analyzed as singlets because of the low sample amount. A total of 150 μL sample was placed in a 2 mL vial along with 50 μL demineralized water, 200 μL ethanol, 40 μL sodium hydroxide (5 w/w %), and 40 μL pyridine, which was done manually. The vials were then placed on a sample tray and the derivatization was performed by a dual rail multi-purpose-sampler (MPS, Gerstel). Internal standard (10 μg ethoxyacetic acid) was added to all vials before each vial was derivatized and extracted one sample at a time. Reagent (2×20 μL) was added twice with vigorous agitation (30 sec) after each addition. Derivatives were extracted by adding 400 μL chloroform containing internal standard (heptadecane, 155 μg/mL) and shaking vigorously for 10 s followed by addition of 400 μL sodium bicarbonate (50 mM) and vigorous agitation. All organic extracts were transferred and dried in batches by slowly aspirating 200 μL of the bottom organic phase to vials with inserts containing anhydrous sodium sulphate.

Derivatized samples were placed in sample trays (98 position trays) with cooling (5° C.) until being analyzed with GC-TOFMS (Agilent 7890, LECO Pegasus® HT). The GC was equipped with an Rtx5-MS (Restek, 30 m×0.25 mm×0.25 μm). The inlet was operated in split mode (1:20) at 280° C. The injection volume was 1 μL. Helium was used as carrier gas at 1 mL/min. The oven temperature program was started at 50° C. and increased 10° C./min to 320° C. (hold 10 min) giving a total run time of 37 min. The transfer line was 250° C. The ion source temperature was 250° C., the acquisition rate was 20 hz, and the mass range was 25-1000 m/z.

A characteristic ion of each analyte was selected and extracted as the response, which was normalized with the response of the internal standard. Specifically, the characteristic ions of pimelic acid (ethyl derivative) and azelaic acid (ethyl derivative) were 125 m/z and 83 m/z, respectively. A pooled control sample was made by taking an aliquot from all samples. The pooled control was derivatized and analysed for every 15 injections of sample. The response of the pooled control was used for drift correction of the instrument performance. Relative response of pimelic acid and azelaic acid were obtained by dividing with the total peak area of the specific sample.

### Experiment—Design & Parameters

The basic design for the responder analysis experiment is a cross-section balanced Control-Treatment setup (status=ITT), with subject longitudinal observations at 2 time points (v2=baseline, v5=after 6 months intervention) and the control equal to Placebo and the treatment equal to B420.

The experiment is a multi-compartment trial, sharing the same basic design in different versions, and with the clinical compartment as the inner (core) array and the other compartments as outer (manifestation) arrays.

In tablet below is outlined the 12 compartments in the experiment with summary data of number of subjects (N_Subj) and number of parameters within each compartment (N_Param). Compartment #1 is the clinical compartment and compartments #2-12 are different manifestation compartments.

TABLE 1
The compartment structure of the study.

| _COMPART_{—} | _TYPE_{—} | _TYPE2_{—} | _TYPE3_{—} | N_Subj | N_Param |
|---|---|---|---|---|---|
| 1 | CLIN | Clin | Clin | 355 | 114 |
| 2 | CLIN | Feces | Clin | 106 | 2 |
| 3 | GCMS | Feces | Metabol | 106 | 86 |
| 4 | Genomics | Feces | AlphaDiversity/Taxa | 106 | 441 |
| 5 | LCMS | Feces | BA | 106 | 21 |
| 6 | LCMS | Serum | BA | 106 | 17 |
| 7 | LCMS | Serum | Lipid_AcCar | 87 | 19 |
| 8 | LCMS | Serum | Lipid_ESN | 87 | 394 |
| 9 | LCMS | Serum | Lipid_ESP | 87 | 1359 |
| 10 | NMR | Feces | Metabol | 106 | 31 |
| 11 | NMR | Serum | Glyc | 355 | 2 |
| 12 | NMR | Serum | Metabol | 355 | 47 |

The clinical compartment covers the effective ITT population of the general clinical trial, and the other compartments are different subsets of the clinical compartment population.

Responder status is based on 2 clinical parameters as outlined in table2 below (_RESP_is the responder status classifier).

TABLE 2
Responder status classifiers.

| _NAME_{—} | _NAME2_{—} | _RESP_{—} | Responder status |
|---|---|---|---|
| DXAFMASS | Total Body (g) | DXAFMASS_5% | “Responder” if the corresponding (v5-v2) value |
| | | | (relative) is <−5% or else “non-Responder”. Thus, |
| | | | responders have at least a 5% reduction in total fat |
| GLUC | Glucose (mmol/L) | GLUC | “Responder” if the corresponding (v5-v2) value |
| | | | (absolute) is <0 or else “non-Responder”. |

Thus, 2 different responder status classifiers (DXAFMASS_5%, GLUC) are used through the post hoc analysis and they are all defined by the contrast during the intervention period (v5-v2).

The objective is to identify parameters/biomarkers, based on baseline (v2) values for the whole ITT population, that significantly discriminate between B420 and Placebo for responders and are significantly different from that of non-responders.

### Model

A multivariate latent class model (LCA) is fitted to the data using the clinical compartment as inner (core) array and the other compartment (manifestations) as outer arrays, using the statistical software SAS 9.4 (SAS Institute Inc., Cary, NC, USA, 2016).

In the model is used the 2 classification identifiers, treatment (B420, Placebo) and responder status (non-responders, responders), in a regression setup for both intercept and slopes.

The associations between a responder status classifier and potential biomarkers are summarised by a set of 3 information carriers (t, t_0, t_1), being the difference (B420 vs Placebo) in slopes for respectively the prospective setup (without knowing the responder status) and the retrospective setup for non-responders and responders.

The stats are information carriers in Z-scale, and these are interpreted by the sign (the direction of the effect) and the size (the strength such that higher absolute value is more significant).

Significant biomarkers at level 5% by a 2-sided test (i.e. an absolute Z-score >1.96) are indicated by colour codes (red=positive score for information carrier, blue=negative score for information carrier).

### LCA Model Results

Below, is summarised significant biomarkers for the responder status classifier (DXAFMASS) in table 3 and the responder status classifier (GLUC) in table 4.

At the left is the biomarker characteristics; in the ANOVA-part is the information carriers; in the THRESHOLD-part is the threshold for the biomarkers in the B420 population (MEDIAN) and the P33 and P66 percentiles.

TABLE 3
Biomarkers (Z-scores, column t, t_{——}0, t_{——}1) for the responder status classifier (DXAFMASS).

| | | | | | ANOVA | | | THRESHOLD | | |
|---|---|---|---|---|---|---|---|---|---|---|
| _TYPE_{—} | _TYPE2_{—} | _TYPE3_{—} | _NAME_{—} | _RESP_{—} | t | t_{——}0 | t_{——}1 | MEDIAN | P33 | P66 |
| LCMS | Serum | Lipid_ESP | PC(36:4) | DXAFMASS_5% | −3.55 | −1.02 | −3.60 | 0.063777 | 0.056972 | 0.066404 |
| LCMS | Serum | Lipid_ESP | PC(32:1) | DXAFMASS_5% | −3.44 | −0.90 | −3.52 | 0.00369 | 0.003374 | 0.004155 |
| LCMS | Serum | Lipid_ESP | PE(38:4) | DXAFMASS_5% | −3.34 | −0.06 | −4.47 | 0.00074 | 0.000651 | 0.000806 |
| LCMS | Serum | Lipid_ESP | SM(d40:1) | DXAFMASS_5% | −3.16 | −0.76 | −2.91 | 0.010681 | 0.009959 | 0.011270 |
| LCMS | Serum | Lipid_ESN | PE(36:4) | DXAFMASS_5% | −3.11 | 0.09 | −4.32 | 0.00091 | 0.000783 | 0.001039 |
| LCMS | Serum | Lipid_ESN | PI(40:4) | DXAFMASS_5% | −2.63 | 0.60 | −4.26 | 0.000191 | 0.000172 | 0.000214 |
| LCMS | Serum | Lipid_ESN | PE(40:5) | DXAFMASS_5% | −2.63 | 0.20 | −3.69 | 0.000157 | 0.000139 | 0.000239 |
| LCMS | Feces | BA | ISO-LCA | DXAFMASS_5% | −3.46 | −1.58 | −3.09 | 29.40213 | 19.38917 | 38.0078 |
| LCMS | Feces | BA | LCA | DXAFMASS_5% | −2.44 | −0.86 | −2.43 | 1540.06 | 1216.08 | 1997.38 |
| Genomics | Feces | Taxa | Aminipila | DXAFMASS_5% | −2.36 | 0.16 | −2.83 | 0.000433 | 0.000000 | 0.000686 |
| | | | butyrica | | | | | | | |

TABLE 4
Biomarkers (Z-scores, column t, t_{——}0, t_{——}1) for the responder status classifier (GLUC).

| | | | | | ANOVA | | | THRESHOLD | | |
|---|---|---|---|---|---|---|---|---|---|---|
| _TYPE_{—} | _TYPE2_{—} | _TYPE3_{—} | _NAME_{—} | _RESP_{—} | t | t_{——}0 | t_{——}1 | MEDIAN | P33 | P66 |
| Genomics | Feces | Taxa | Unclassified | GLUC | −2.88 | −1.80 | −2.31 | 0.000185 | 7E−06 | 0.000527 |
| | | | Clostridiales_{—} | | | | | | | |
| | | | Incertae Sedis | | | | | | | |
| | | | XIII | | | | | | | |
| Genomics | Feces | Taxa | Eubacterium | GLUC | −2.46 | −1.60 | −1.89 | 0.008469 | 0.006413 | 0.010646 |
| | | | coprostanoligenes | | | | | | | |
| GCMS | Feces | Metabol | Pimelic acid | GLUC | −3.43 | −0.57 | −4.21 | 0.014266 | 0.000789 | 0.033695 |
| GCMS | Feces | Metabol | Azelaic acid | GLUC | −3.21 | −0.15 | −4.23 | 0.012386 | 0.009495 | 0.017131 |
| CLIN | Clin | STP | AVGSTEPS | GLUC | −2.22 | −0.82 | −2.45 | 7017 | 5600.76 | 8806.6 |
| LCMS | Serum | BA | LCA nmol/mL | GLUC | −2.06 | −0.68 | −2.23 | 0.010658 | 0.005329 | 0.01471 |
| LCMS | Serum | BA | DCA nmol/mL | GLUC | −2.05 | 0.59 | −3.26 | 0.406035 | 0.280146 | 0.521839 |

For both responder status classifiers (DXAFMASS, GLUC) are observed that the biomarker in high value increases the probability of being a responder (prospectively) and this is retrospectively confirmed by “t_1” being significant and “t_0” being non-significant.

| Abbreviation | | | |
|---|---|---|---|
| Term | Class | Name/identifier | Structural details/links to webpages: |
| PC (36:4) | Phospholipid | Phosphatidylcholine | Exact structure PC (16:0_20:4): |
| | (Lipid_ESP*) | (36:4) | https://www.lipidmaps.org/databases/lmsd/LMGP01010629 |
| | | Monoisotopic | Systematic name: |
| | | mass: m/z: | 1-hexadecanoyl-2-(5E,8E,11E,14E- |
| | | 781.5622 | eicosatetraenoyl)-sn-glycero-3- |
| | | Molformula: | phosphocholine |
| | | C44H80N1O8P1 | |
| PC (32:1) | Phospholipid | Phosphatidylcholine | Exact structure PC (16:0_16:1) |
| | (Lipid_ESP*) | (32:1) | https://www.lipidmaps.org/databases/lmsd/LMGP01010566 |
| | | Monoisotopic | Systematic name: |
| | | mass: | 1-hexadecanoyl-2-(9Z-hexadecenoyl)-sn- |
| | | m/z: 731.5465 | glycero-3-phosphocholine |
| | | Molformula: | |
| | | C40H78N1O8P1 | |
| PE (38:4) | Phospholipid | Phosphatidyl- | Exact structure PE (18:0_20:4) |
| | (Lipid_ESP*) | ethanolamine | Observed mass: 768.5538 [M + H]+ |
| | | (38:4) | (See example below on assignment structure). |
| | | Monoisotopic | https://www.lipidmaps.org/databases/lmsd/LMGP02010129 |
| | | mass: m/z: | 1-octadecanoyl-2-(5E,8E,11E,14E- |
| | | 767.5465 | eicosatetraenoyl)-sn-glycero-3- |
| | | Molformula: | phosphoethanolamine |
| | | C43 H78 N1 O8 P1 | |
| SM (d40:1) | Phospholipid | Sphingomyelin | Exact structure SM (d40:1), can be either: |
| | (Lipid_ESP*) | Monoisotopic | https://www.lipidmaps.org/databases/lmsd/LMSP03010006 |
| | | mass: | N-(docosanoyl)-sphing-4-enine-1- |
| | | m/z: 786.6615 | phosphocholine |
| | | Molformula: | or: |
| | | C45 H91 N2 O6 P1 | https://www.lipidmaps.org/databases/lmsd/LMSP03010073 |
| | | | N-(tetracosanoyl)-hexadecasphing-4-enine-1- |
| | | | phosphocholine |
| PE (36:4) | Phospholipid | Phosphatidyl- | Exact structure PE (16:0_20:4) |
| | (Lipid_ESN**) | ethanolamine(36:4) | https://www.lipidmaps.org/databases/lmsd/LMGP02010096 |
| | | Monoisotopic | 1-hexadecanoyl-2-(5Z,8Z,11Z,14Z- |
| | | mass: | eicosatetraenoyl)-sn-glycero-3- |
| | | m/z: 739.5152 | phosphoethanolamine |
| | | Molformula: | |
| | | C41 H74 N1 O8 P1 | |
| PI (40:4) | Phospholipid | Phosphatidyl- | Exact structure PI (18:0_22:4) |
| | (Lipid_ESN**) | inositol (40:4) | https://www.lipidmaps.org/databases/lmsd/LMGP06010853 |
| | | Monoisotopic | 1-octadecanoyl-2-(7Z,10Z,13Z,16Z- |
| | | mass: | docosatetraenoyl)-glycero-3-phospho-(1′- |
| | | m/z: 914.5884 | myo-inositol) |
| | | Molformula: | |
| | | C49 H87 O13 P1 | |
| PE (40:5) | Phospholipid | Phosphatidyl- | Exact structure PE (18:1_22:4) |
| | (Lipid_ESN**) | ethanolamine(40:5) | https://www.lipidmaps.org/databases/lmsd/LMGP02010652 |
| | | Monoisotopic | 1-(9Z-octadecenoyl)-2-(7Z,10Z,13Z,16Z- |
| | | mass: m/z = | docosatetraenoyl)-glycero-3- |
| | | 793.5622 | phosphoethanolamine |
| | | Molformula: | |
| | | C45 H80 N1 O8 P1 | |
| LCA | Bile acid | Lithocholic acid | Secondary Bile acid (BA); unconjugated |
| | (BA) | Monoisotopic | https://hmdb.ca/metabolites/HMDB0000761 |
| | | mass: m/z = | |
| | | 376.2977 | |
| | | Mw: 376.5725 | |
| | | g/MOL | |
| | | CAS No: 434- | |
| | | 13-9 | |
| | | C₂₄M₄₀O₃ | |
| Iso-LCA | Bile acid | Iso-Lithocholic | Secondary Bile acid (BA); unconjugated |
| | (BA) | acid | https://hmdb.ca/metabolites/HMDB0000717 |
| | | Monoisotopic | |
| | | mass: m/z = | |
| | | 376.2977 | |
| GLCA | Bile acid | Glycolithocholic | Glycine conjugated Lithocholic acid |
| | (BA) | acid: | https://hmdb.ca/metabolites/HMDB0000698 |
| | | Glycine | |
| | | conjugated | |
| | | Lithocholic acid | |
| | | Monoisotopic | |
| | | mass: m/z = | |
| | | 433.3192 | |
| | | Mw: | |
| | | 433.6239 g/mol | |
| | | CAS No: 474- | |
| | | 74-8 | |
| | | C₂₆H₄₃NO₄ | |
| DCA | Bile acid | Deoxycholic | Secondary Bile acid (BA); unconjugated |
| | (BA) | acid | https://hmdb.ca/metabolites/HMDB0000626 |
| | | Monoisotopic | |
| | | mass: m/z = | |
| | | 392.2926 | |
| | | Mw: | |
| | | 392.572 g/mol | |
| | | CAS No: | |
| | | C₂₄H₄₀O₄ | |
| Azelaic | Metabolites | Nonanedioic | https://hmdb.ca/metabolites/HMDB0000784 |
| acid | Dicarboxylic | acid | |
| | acid | CAS No: 123- | |
| | | 99-9 | |
| Pimelic | Metabolites | Heptanedioic | https://hmdb.ca/metabolites/HMDB0000857 |
| acid | Dicarboxylic | acid | |
| | acid | CAS No: 11- | |
| | | 16-0 | |
| hPAI1 | Clinical | Total human | https://www.ncbi.nlm.nih.gov/pmc/articles/PMC2958211/ |
| | | Plasminogen | |
| | | activator | |
| | | inhibitor-1 | |

*)Detected in LC-MS mode: Electrospray Positive mode;
**)Detected in LC-MS mode: Electrospray Negative mode.

Exact structure assignment of Phospholipids was based on high resolution LC-MS-MS fragmentation.

### REFERENCES

Amabebe et al. 2020
Amabebe E, Robert F O, Agbalalah T, Orubu E S F. Microbial dysbiosis-induced obesity: role of gut microbiota in homoeostasis of energy metabolism. Br J Nutr. 2020 May 28; 123(10):1127-1137. doi: 10.1017/S0007114520000380. Epub 2020 Feb. 3. PMID: 32008579.
Waterson and Horvath 2015
Waterson M J, Horvath T L. Neuronal Regulation of Energy Homeostasis: Beyond the Hypothalamus and Feeding. Cell Metab. 2015 Dec. 1; 22(6):962-70. doi: 10.1016/j.cmet.2015.09.026. Epub 2015 Oct. 22. PMID: 26603190.
Stenman et al. 2016
Lotta K. Stenman, Markus J. Lehtinen, Nils Meland, Jeffrey E. Christensen, Nicolas Yeung, Markku T. Saarinen, Michael Courtney, Rémy Burcelin, Marja-Leena Lähdeaho, Jüri Linros, Dan Apter, Mika Scheinin, Hilde Kloster Smerud, Aila Rissanen, Sampo Lahtinen. Probiotictic With or Without Prebiotic Controls Body Fat Mass, Associated With Serum Zonulin, in Overweight and Obese Adults-Randomized Controlled Trial. Ebiomedicine (2016) 13:190-200. Doi: 10.1016/j.ebiom.2016.10.036.
Hibberd et al. 2019
Hibberd, A. A., Yde, C. C., Ziegler, M. L., Honore, A. H., Saarinen, M. T., Lahtinen, S., Stahl, B., Jensen, H. M., and Stenman, L. K. (2019). Probiotic or synbiotic alters the gut microbiota and metabolism in a randomised controlled trial of weight management in overweight adults. Beneficial microbes, 2019; 10(2): 121-135. Doi: 10.3920/BM2018.0028.
Satomi Y. et al. 2017
One-step lipid extraction for plasma lipidomics analysis by liquid chromatography mass spectrometry. Satomi Y., Hirayama M., Kobayashi H. (2017) Journal of Chromatography B: Analytical Technologies in the Biomedical and Life Sciences, 1063, pp. 93-100.
Cajka T and Fiehn O 2014
Cajka T, Fiehn O (2014) Comprehensive analysis of lipids in biological systems by liquid chromatography-mass spectrometry. Trac-Trend Anal Chem 61:192-206.
Sanjoy K. Bhattacharva 2017 Sanjoy K. Bhattacharya (ed.), Lipidomics: Methods and Protocols, Methods in Molecular Biology, vol. 1609, DOI 10.1007/978-1-4939-6996-8_14, © Springer Science+Business Media LLC 2017 (Chapter 14).
Zhao L, et al 2017
Zhao L, et al (2017) High Throughput and Quantitative Measurement of Microbial Metabolome by Gas Chromatography/Mass Spectrometry Using Automated Alkyl Chloroformate Derivatization. DOI 10.1021/acs.analchem.7b00660, Anal. Chem. 2017, 89, 10, 5565-5577

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### Taxonomy

As used herein the term “Aminipila butyrica” refers to the bacterial species of the following taxonomy: Bacteria; Firmicutes; Clostridia; Clostridiales; Clostridiales; Incertae Sedis XIII; Aminipila; Aminipila butyrica

As used herein the term “Clostridiales-Incertae Sedis XIII” refers to the bacterial species of the following taxonomy: Bacteria; Firmicutes; Clostridia; Clostridiales; Clostridiales; Incertae Sedis XIII; Unclassified Clostridiales Incertae Sedis XIII.

As used herein the term “Eubacterium coprostanoligenes” refers to the bacterial species of the following taxonomy: Bacteria; Firmicutes; Clostridia; Clostridiales; Eubacteriaceae; Eubacterium; Eubacterium coprostanoligenes.

As used herein the term “Coprococcus” refers to the genus of anaerobic cocci including the species Coprococcus catus, Coprococcus comes, and Coprococcus eutactus.

As used herein the term “Ruminococcus” refers to the genus of bacteria in the class Clostridia including the species selected from Ruminococcus albus, Ruminococcus bromii, Ruminococcus callidus, Ruminococcus flavefaciens Ruminococcus gauvreauii, Ruminococcus gnavus, Ruminococcus lactaris, Ruminococcus obeum, and Ruminococcus torques.

1. A method of identifying a subject having an increased probability of having a beneficial clinical response to administration of a bacterial strain of the genus Bifidobacterium or a mixture of two or more strains thereof, the method comprising the steps of,
i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps or corresponding physical activity; and
ii. comparing said level to a threshold value,
wherein when the measured level of at least one of the biomarkers is higher than said threshold value, the subject is identified as having said increased probability.

2. The method according to claim 1, wherein the beneficial clinical response to said bacterial strain of the genus Bifidobacterium or a mixture of two or more said strains thereof is at least one of weight management, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in said subject.

3. The method according to any one of the preceding claims, wherein said bacterial strain of the genus Bifidobacterium or a mixture of two or more said strains thereof if of the species Bifidobacterium animalis, preferably of the species Bifidobacterium animalis subsp. Lactis, such as the bacterial strain of the species Bifidobacterium animalis subsp. Lactis strain B420.

4. The method according to any one of the preceding claims, wherein the measured level of hPAI1 Total in the biological sample obtained from said subject is above 48245 pg/ml.

5. The method according to any one of the claims 1-4, wherein the measured level of GLCA in the biological sample obtained from said subject is above 0.0070 μmol/l and/or the measured level of LCA in the biological sample obtained from said subject is above 0.0380 μmol/l.

6. The method according to any one of the claims 1-5, wherein the measured level of relative abundance of Coprococcus in the biological sample obtained from said subject is above 0.0426 and/or the measured level of relative abundance of Ruminococcus in the biological sample obtained from said subject is above 0.0631 and/or the measured level of relative abundance of Akkermansia in the biological sample obtained from said subject is above 0.0062 and/or the measured level of relative abundance Aminipila butyrica in the biological sample obtained from said subject is above 0.00043 and/or the measured level of relative abundance Unclassified Clostridiales-Incertae Sedis XIII in the biological sample obtained from said subject is above 0.00018 and/or the measured level of relative abundance Eubacterium coprostanoligenes in the biological sample obtained from said subject is above 0.0084.

7. The method according to any one of the claims 1-6, wherein the measured level of Iso-LCA in the biological sample, such as in feces, obtained from said subject is above 29 μmol/l and/or the measured level of DCA in the biological sample, such as in blood serum, obtained from said subject is above 0.41 nmol/ml.

8. The method according to any one of the claims 1-7, wherein the measured level of the phospholipid phosphatidylcholine(36:4) in the biological sample obtained from said subject measured as relative amount calculated as peak area for the phospholipid divided with the total peak area of all identified lipids in positive ionization mode as described herein is above 0.064 and/or the measured level of the phospholipid phosphatidylcholine(32:1) in the biological sample obtained from said subject measured as relative amount calculated as peak area for the phospholipid divided with the total peak area of all identified lipids in positive ionization mode as described herein is above 0.0037 and/or the measured level of the phospholipid phosphatidylethanolamine(38:4) in the biological sample obtained from said subject measured as relative amount calculated as peak area for the phospholipid divided with the total peak area of all identified lipids in positive ionization mode as described herein is above 0.00074 and/or the measured level of the phospholipid phosphatidylethanolamine(36:4) in the biological sample obtained from said subject measured as relative amount calculated as peak area for the phospholipid divided with the total peak area of all identified lipids in negative ionization mode as described herein is above 0.00091 and/or the measured level of the phospholipid phosphatidylinositol(40:4) in the biological sample obtained from said subject measured as relative amount calculated as peak area for the phospholipid divided with the total peak area of all identified lipids in negative ionization mode as described herein is above 0.00019.

9. The method according to any one of the claims 1-8, wherein the measured level of the pimelic acid in the biological sample obtained from said subject measured as relative amount calculated as peak area of ion 125 m/z divided with the total peak area of all identified peaks as described herein is above 0.000126 and/or the measured level of the azelaic acid in the biological sample obtained from said subject measured as relative amount calculated as peak area of ion 83 m/z divided with the total peak area of all identified peaks as described herein is above 0.000135.

10. The method according to any one of the preceding claims, wherein the daily activity of the subject in steps is above 7000, or corresponding physical activity.

11. Use of a bacterial strain of the genus Bifidobacterium or a mixture of two or more strains thereof for at least one of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject, wherein said subject has been identified as having an increased probability of having a beneficial clinical response to administration of said bacterial strain of the genus Bifidobacterium or a mixture of two or more strains thereof, by,
i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps; and
ii. comparing said level to a threshold value,
wherein when the measured level of at least one of the biomarkers is higher than said threshold value, the subject is identified as having said increased probability.

12. The use of a bacterial strain of the genus Bifidobacterium or a mixture of two or more strains according to claim 11 as further defined in any one of the claims 1-10.

13. Bacterial strain of the genus Bifidobacterium or a mixture of two or more strains thereof for use in managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject, wherein said subject has been identified as having an increased probability of having a beneficial clinical response to administration of said bacterial strain of the genus Bifidobacterium or a mixture of two or more strains thereof, by,
i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps; and
ii. comparing said level to a threshold value,
wherein when the measured level of at least one of the biomarkers is higher than said threshold value, the subject is identified as having said increased probability.

14. The bacterial strain of the genus Bifidobacterium or a mixture of two or more strains for use according to claim 13 as further defined in any one of the claims 1-10.

15. Method of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject in need thereof, said method comprising administering a bacterial strain of the genus Bifidobacterium or a mixture of two or more strains thereof to said subject, wherein said subject has been identified as having an increased probability of having a beneficial clinical response to the administration of said bacterial strain of the genus Bifidobacterium or a mixture of two or more strains thereof, by,
i. measuring the level of at least one of the following biomarkers: hPAI1 Total; a bile acid selected from GLCA, LCA, Iso-LCA, and DCA; a phospholipid selected from phosphatidylcholine(36:4), phosphatidylcholine(32:1), phosphatidylethanolamine(38:4), phosphatidylethanolamine(36:4), and phosphatidylinositol(40:4); a bacteria selected from Coprococcus, Ruminococcus, Akkermansia, Aminipila butyrica, Unclassified Clostridiales-Incertae Sedis XIII, and Eubacterium coprostanoligenes; Pimelic acid and Azelaic acid; in a biological sample obtained from said subject, and/or the daily activity of the subject in steps; and
ii. comparing said level to a threshold value,
wherein when the measured level of at least one of the biomarkers is higher than said threshold value, the subject is identified as having said increased probability.

16. The method according to claim 15 as further defined in any one of the claims 1-10.

17. A method of managing weight, such as, lowering BMI, lowering blood glucose level, reducing body fat, controlling weight gain, inducing weight loss, lowering body fat mass, lowering mesenteric fat mass, lowering trunk fat mass and/or decreasing android fat mass, treating diabetes (preferably but not exclusively Type 2 diabetes), treating impaired glucose tolerance, normalizing insulin sensitivity, increasing fed insulin secretion, decreasing fasted insulin secretion, improving glucose tolerance, treating obesity, lowering tissue inflammation (particularly, although not exclusively, muscle tissue inflammation, liver tissue inflammation and/or adipose tissue inflammation), treating hepatitis, treating myositis, treating cardiovascular disease and treating metabolic syndrome, in a subject in need thereof, said method comprising
i. Prescribing to the subject a daily personal activity of more than 7000 steps, or corresponding physical activity; and
ii. administering a bacterial strain of the genus Bifidobacterium or a mixture of two or more strains thereof to the subject having obtained the activity under i), which subject has an increased probability of having a beneficial clinical response to the administration of said bacterial strain of the genus Bifidobacterium or a mixture of two or more strains thereof.

18. The method according to claim 17, which bacterial strain of the genus Bifidobacterium or a mixture of two or more strains thereof is defined in claim 3.
